(19) United States
(12) Patent Application Publication
(10) Pub. No.: US 2024/0298669 A1
(43) Pub. Date: Sep. 12, 2024

(54) ORALLY DISSOLVING PROTEIN EXTRUDATES

(71) Applicant: CORNELL UNIVERSITY, Ithaca, NY (US)

(72) Inventors: Syed S.H. RIZVI, Ithaca, NY (US); Bindvi ARORA, Ithaca, NY (US)

(73) Assignee: CORNELL UNIVERSITY, Ithaca, NY (US)

(21) Appl. No.: 18/269,225

(22) PCT Filed: Dec. 23, 2021
(86) PCT No.: PCT/US21/65149

§ 371 Date: Jun. 22, 2023

Related U.S. Application Data
(60) Provisional application No. 63/130,250, filed on Dec. 23, 2020.

(51) Int. Cl. A23J 3/26; A23J 1/20; A23J 3/08; A23L 29/00; A23L 29/10; A23L 29/30; A61K 35/20; A61K 47/02

(52) U.S. Cl. CPC A23J 3/26; A23J 1/20; A23J 3/08; A23L 29/015; A23L 29/10; A23L 29/30; A61K 35/20; A61K 47/02

(57) ABSTRACT

Disclosed are orally dissolving protein extrudates, methods of making the orally dissolving protein extrudates, and edible foods containing the orally dissolving protein extrudates. The orally dissolving protein extrudates can contain milk protein concentrate and be made using supercritical fluid extrusion. The supercritical fluid extrusion can be with a dispersing agent that is a calcium chelating agent such as sodium hexametaphosphate.

### CROSS-REFERENCE TO RELATED APPLICATIONS

This application claims priority benefit of U.S. Provisional Patent Application Ser. No. 63/130,250, filed Dec. 23, 2020, the disclosure of which is hereby incorporated by reference herein in its entirety.

### FIELD

The present disclosure relates to, inter alia, orally dissolving protein extrudates, methods of making the orally dissolving protein extrudates, and edible foods containing the orally dissolving protein extrudates.

### BACKGROUND

In the consumer food industry, there is a need for orally dissolving foods that can be consumed by weaning infants, toddlers, the elderly, and dysphagic patients.

Regarding weaning infants and toddlers, the weaning period is a crucial time in an infant's life since it not only involves a great deal of rapid change for the child, but is also associated with the development of food preferences, eating behaviors and body weight in childhood, adolescence as well as in adulthood (Cameron et al., 2012). The recent trend to follow the baby-led weaning (BLW) approach has brought new attention to early practices for solid food introduction (Rapley, 2011). BLW advocates more than simply providing soft solid foods as first foods. It encourages a family mealtime and a healthy approach to feeding infants. While in the traditional weaning infants are offered puréed infant foods, in the BLW approach a variety of single picked foods are offered to the baby. BLW approach might provide an early and more stable learning about the satiating capacities of foods and therefore it may enable a better satiety responsiveness (Brown & Lee, 2015). In the recent past, quick disintegrating puffed cereals have become a popular first finger foods for toddlers that offer the convenience of self-feeding by the child, portability, and mess-free eating environment. These foods claim to have a smooth, easy-to-swallow texture and a simple flavor that is great for babies as they start to explore solid foods (WHO, 2019). The final swallow-safe bolus should be soft, homogenous in texture, cohesive, and slippery enough to allow easy initiation of swallowing and swift transport through the pharynx (Loret et al., 2011; Motoi et al., 2013). These expanded (porous) and quick disintegrating baby puffs are generally prepared by extrusion cooking (WHO, 2019). Quick in-mouth disintegration is an important characteristic of these recently introduced, convenient baby foods which is predominantly achieved by starches.

Therefore, there is a need to develop protein-rich baby foods with solid-like textures that break down and dissolve easily with minimal chewing. The protein-rich texture modified solid foods can serve as a promising alternative to currently available starch-based finger foods. Furthermore, the same protein-rich and orally dissolving foods suitable for baby foods are also suitable for use by the elderly and dysphagic patients, or any other person desiring protein-rich foods that dissolve in-mouth with minimal or no required chewing. There is a particular need for fast orally dissolving foods that are also protein-rich.

The present disclosure is directed to overcoming these and other deficiencies in the art.

### SUMMARY

The present disclosure relates to, inter alia, orally dissolving protein extrudates, methods of making the orally dissolving protein extrudates, and edible foods containing the orally dissolving protein extrudates. In a particular embodiment, the orally dissolving protein extrudates contains milk protein concentrate. In a particular embodiment, the orally dissolving protein extrudates can contain milk protein concentrate and be made using supercritical fluid extrusion. The supercritical fluid extrusion can be with a dispersing agent that is a calcium chelating agent, including, for example, sodium hexametaphosphate. In certain embodiments, the orally dissolving protein extrudates of the present disclosure are also, surprisingly, orally dissolving protein extrudates that are protein-rich, fast orally dissolving protein extrudates, or fast orally dissolving protein extrudates that are protein-rich.

In one aspect, the present disclosure provides to a method of preparing an orally dissolving protein extrudate product, the method comprising: (a) blending a protein concentrate with one or more extrusion component to produce a protein dough; (b) extruding the protein dough with supercritical fluid to form a wet protein extrudate, said extruding being effective to inhibit protein-protein interactions in the protein concentrate; and (c) drying the wet protein extrudate to form a protein extrudate product that is orally dissolving. Also provided are orally dissolving protein extrudate products prepared by this method.

In another aspect, the present disclosure provides a protein extrudate product comprising: (a) a protein concentrate; (b) a sugar; (c) an emulsifier; and (d) a dispersing agent comprising a calcium chelating agent, wherein the protein extrudate product is orally dissolving and protein-rich. In one embodiment, the protein extrudate product includes: (a) protein concentrate at about 60-90 wt %, or at about 65-85 wt %, or at about 84 wt %; (b) sugar at about 5-25 wt %, or at about 10-20 wt %, or at about 13.5 wt %; (c) emulsifier(s) at about 0.05-4 wt %, or at about 1-3 wt %, or at about 1-2 wt %; and (d) dispersing agent at about 0.1-0.5 wt %, or at about 0.3-0.5 wt %, or at about 0.4-0.5 wt %. In another embodiment, the protein concentrate comprises a milk protein concentrate. Also provided are edible foods comprising this protein extrudate product.

As disclosed herein, in particular embodiments, the supercritical fluid extrusion uses carbon dioxide as the supercritical fluid (suitable to temporarily lower the pH during extrusion) along with sodium hexametaphosphate (SHMP) as the dispersing agent. This combination surprisingly yields orally dissolving and fast orally dissolving protein extrudates that are protein-rich. The method of the present disclosure is advantageous over the existing art in that it does not require the addition of acidifying agents such as phosphoric acid and/or citric acid, which are likely to leave acidic taste in the finished products unless neutralized by adding alkaline agents. Another advantage of the method of the present disclosure over the existing art is that it does not require high temperatures, whereas some existing processes for protein extrusion in the food processing industry require the use of conventional, steam-based extrusion with processing temperatures around 200° C.

Unlike preexisting protein extrusion techniques used in the food processing industry, in certain embodiments, the method of the present disclosure involves low temperature (<100° C.) supercritical fluid extrusion where acidity is induced temporarily within the extruder (in situ) due to dissolution of carbon dioxide in water and formation of carbonic acid, with SHMP acting as a calcium chelating agent. Once the protein extrudate product is extruded, there is little to no remaining acidity in the product. Thus, there are little to no acidifying agents or their residues in the end product of the method of the present disclosure.

Furthermore, the method of the present disclosure is advantageous over the existing art in that the lower processing temperature during SCFX (<100° C.) prevents damage to the protein structure, protein polymerization as well as browning reactions (Maillard reaction), and contributes in the quick dissolution characteristics of the developed product of the present disclosure. In certain embodiments, one major advantage of the method of the present disclosure is that the end product has a fast orally dissolving quality of a protein-rich puffed extrudate.

In particular embodiments, the present disclosure provides milk-based puffs prepared by SCFX. Some advantages of these embodiments over the existing art include, without limitation, the following:
(a) Ready to eat and do not require reconstitution by water addition: They provide the convenience of self-feeding by the child, portability and provide mess-free eating environment.
(b) Quick, in-mouth disintegrating (in <30 seconds): They have quick disintegration ability and its structure collapses in the mouth in less than 30 seconds. The product does not present any choking hazard to toddlers.
(c) Milk protein-based with complete amino acid profile: They are made of milk protein concentrate and have at least 65% protein (final product).
(d) Good in nutritional and organoleptic properties: Milk proteins are a source of nitrogen and amino acids which are essential for the growth of the neonate, toddler, child and for the maintenance of various bodily functions. The puffs are sweet tasting and have good sensorial acceptability.
(e) Shelf-stable: They are dry packaged and may have a shelf life of more than a year.
(f) Storable after partial consumption: They can be marketed in re-sealable packaging and can be stored after opening for later consumption.
(g) Lightweight and easy to transport: They are expanded puffs making them extremely light and easy to carry and transport.
(h) The milk protein puffs may have other uses, e.g., for elderly and dysphagia patients or patients with other conditions that make chewing and swallowing difficult.

Various aspects of the present invention are also addressed by the following Paragraphs 1-55 and in the noted combinations thereof, as follows:

Paragraph 1: A method of preparing an orally dissolving protein extrudate product, the method comprising: (a) blending a protein concentrate with one or more extrusion component to produce a protein dough; (b) extruding the protein dough with supercritical fluid to form a wet protein extrudate, said extruding being effective to inhibit protein-protein interactions in the protein concentrate; and (c) drying the wet protein extrudate to form a protein extrudate product that is orally dissolving.

Paragraph 2: The method of Paragraph 1, wherein the protein concentrate comprises milk protein concentrate.

Paragraph 3: The method of any one of Paragraphs 1-2, wherein the one or more extrusion component comprises one or more sugar, emulsifier, and/or dispersing agent.

Paragraph 4: The method of Paragraph 3, wherein the sugar comprises one or more saccharide selected from the group consisting of monosaccharides, disaccharides, and oligosaccharides, wherein the monosaccharides comprise glucose and/or fructose, the disaccharides comprise sucrose and/or maltose, and the oligosaccharides comprise maltodextrins.

Paragraph 5: The method of Paragraph 3, wherein the saccharide is sucrose.

Paragraph 6: The method of Paragraph 3, wherein the emulsifier is selected from the group consisting of lecithin, distilled monoglycerides, polysorbates, and diacetyl tartaric esters of mono and diglycerides.

Paragraph 7: The method of Paragraph 3, wherein the dispersing agent is a calcium chelating agent selected from the group consisting of sodium hexametaphosphate (SHMP), trisodium diphosphate, and tetrasodium diphosphate.

Paragraph 8: The method of Paragraph 3, wherein the dispersing agent is sodium hexametaphosphate (SHMP).

Paragraph 9: The method of any one of Paragraphs 1-8, wherein the protein dough comprises: (a) protein concentrate at about 40-80 wt %, or at about 50-70 wt %, or at about 60 wt %; (b) sugar at about 2-20 wt %, or at about 5-15 wt %, or at about 9.6 wt %; (c) emulsifier(s) at about 0-3 wt %, or at about 0.5-2 wt %, or at about 1.42 wt %; (d) dispersing agent at about 0.1-0.5 wt %, or at about 0.3-0.5 wt %, or at about 0.357 wt %; and (e) water at about 20-40 wt %, or at about 25-35 wt %, or at about 28.5 wt %.

Paragraph 10: The method of any one of Paragraphs 1-9, wherein the protein dough comprises about 840 kg of protein concentrate, about 135 kg of saccharide, about 10 kg of lecithin, about 10 kg of distilled monoglycerides, and about 5 kg of dispersing agents

Paragraph 11: The method of any one of Paragraphs 1-10, wherein the extruding the protein dough with supercritical fluid comprises a feed rate of about 1-50 kg/h.

Paragraph 12: The method of any one of Paragraphs 1-10, wherein the extruding the protein dough with supercritical fluid comprises a feed rate of about 35 kg/h.

Paragraph 13: The method of any one of Paragraphs 1-10, wherein the extruding the protein dough with supercritical fluid comprises a screw speed of about 1-200 rpm.

Paragraph 14: The method of any one of Paragraphs 1-10, wherein the extruding the protein dough with supercritical fluid comprises a screw speed of about 110 rpm.

Paragraph 15: The method of any one of Paragraphs 1-10, wherein the extruding the protein dough with supercritical fluid comprises a supercritical fluid injection pressure of about 1 to 20 MPa.

Paragraph 16: The method of any one of Paragraphs 1-10, wherein the extruding the protein dough with supercritical fluid comprises a supercritical fluid injection pressure of about 11 MPa.

Paragraph 17: The method of any one of Paragraphs 1-10, wherein the extruding the protein dough with supercritical fluid comprises a barrel set temperature of about 5 to 40° C.

Paragraph 18: The method of any one of Paragraphs 1-10, wherein the extruding the protein dough with supercritical fluid comprises supercritical fluid feed rate of about 0.8 kg/h.

Paragraph 19: The method of any one of Paragraphs 1-10, wherein the extruding the protein dough with supercritical fluid comprises a water feed rate of about 14 kg/h.

Paragraph 20: The method of any one of Paragraphs 1-20, wherein the supercritical fluid is carbon dioxide.

Paragraph 21: The method of any one of Paragraphs 1-21, wherein the blending comprises dry blending.

Paragraph 22: The method of Paragraph 21, wherein the dry blending comprises blending in a ribbon blender for about 5-30 minutes, or for about 10, 15, 20, 25, or 30 minutes.

Paragraph 23: The method of any one of Paragraphs 1-22, wherein the wet protein extrudate is collected at a temperature of between about 70-95° C., or about 70° C., 75° C., 80° C., 85° C., 90° C., or 95° C.

Paragraph 24: The method of any one of Paragraphs 1-22, wherein the wet protein extrudate is collected at a temperature of about 85° C.

Paragraph 25: The method of any one of Paragraphs 1-24, wherein the drying comprises conventional oven drying or vacuum oven drying.

Paragraph 26: The method of any one of Paragraphs 1-25, wherein the drying comprises drying until a moisture content of the protein extrudate product is less than about 5-15%, or about 5%, 10%, or 15%.

Paragraph 27: The method of any one of Paragraphs 1-26, wherein the drying comprises conventional drying at greater than about 50-95° C. with or without vacuum, or greater than about 50° C., 60° C., 70° C., 80° C., 85° C., 90° C., or 95° C. with or without vacuum.

Paragraph 28: The method of any one of Paragraphs 1-27, wherein the protein extrudate product is fast orally dissolving.

Paragraph 29: The method of any one of Paragraphs 1-28, wherein the protein extrudate product is protein-rich.

Paragraph 30: An orally dissolving protein extrudate product prepared by the method of any one of Paragraphs 1-29.

Paragraph 31: The protein extrudate product of Paragraph 30 comprising: (a) protein concentrate at about 60-90 wt %, or at about 65-85 wt %, or at about 84 wt %; (b) sugar at about 5-25 wt %, or at about 10-20 wt %, or at about 13.5 wt %; (c) emulsifier(s) at about 0.05-4 wt %, or at about 1-3 wt %, or at about 1-2 wt %; and (d) dispersing agent at about 0.1-0.5 wt %, or at about 0.3-0.5 wt %, or at about 0.4-0.5 wt %.

Paragraph 32: The protein extrudate product of Paragraph 30, wherein the protein extrudate product comprises about 84 wt % milk protein concentrate, about 13.5 wt % sucrose, about 1 wt % distilled monoglycerides, and about 0.5 wt % sodium hexametaphosphate.

Paragraph 33: A protein extrudate product comprising: (a) a protein concentrate; (b) a sugar; (c) an emulsifier; and (d) a dispersing agent comprising a calcium chelating agent, wherein the protein extrudate product is orally dissolving and protein-rich.

Paragraph 34: The protein extrudate product of Paragraph 33 comprising: (a) protein concentrate at about 60-90 wt %, or at about 65-85 wt %, or at about 84 wt %; (b) sugar at about 5-25 wt %, or at about 10-20 wt %, or at about 13.5 wt %; (c) emulsifier(s) at about 0.05-4 wt %, or at about 1-3 wt %, or at about 1-2 wt %; and (d) dispersing agent at about 0.1-0.5 wt %, or at about 0.3-0.5 wt %, or at about 0.4-0.5 wt %.

Paragraph 35: The protein extrudate product of any one of Paragraphs 33-34, wherein the protein concentrate comprises a milk protein concentrate.

Paragraph 36: The protein extrudate product of any one of Paragraphs 33-35, wherein the sugar comprises one or more saccharide selected from the group consisting of monosaccharides, disaccharides, and oligosaccharides, wherein the monosaccharides comprise glucose and/or fructose, the disaccharides comprise sucrose and/or maltose, and the oligosaccharides comprise maltodextrins.

Paragraph 37: The protein extrudate product of any one of Paragraphs 33-36, wherein the saccharide is sucrose.

Paragraph 38: The protein extrudate product of any one of Paragraphs 33-37, wherein the emulsifier is a food grade emulsifier selected from the group consisting of lecithin, distilled monoglycerides, polysorbates, and diacetyl tartaric esters of mono and diglycerides.

Paragraph 39: The protein extrudate product of any one of Paragraphs 33-38, wherein the dispersing agent is a calcium chelating agent selected from the group consisting of sodium hexametaphosphate (SHMP), trisodium diphosphate, and tetrasodium diphosphate.

Paragraph 40: The protein extrudate product of any one of Paragraphs 33-39, wherein the dispersing agent is sodium hexametaphosphate (SHMP).

Paragraph 41: The protein extrudate product of any one of Paragraphs 33-40, wherein the protein concentrate is prepared by ultrafiltration.

Paragraph 42: The protein extrudate product of any one of Paragraphs 33-40, wherein the protein concentrate is prepared by diafiltration.

Paragraph 43: The protein extrudate product of any one of Paragraphs 33-42, wherein the protein extrudate product comprises a shelf life of more than about 3-12 months, or more than about 3, 6, 9, or 12 months.

Paragraph 44: The protein extrudate product of any one of Paragraphs 33-43, wherein the protein extrudate product further comprises one or more of a micronutrient, a coloring agent, a flavoring agent, and/or a food grade additive.

Paragraph 45: The protein extrudate product of Paragraph 44, wherein the one or more micronutrient is selected from the group consisting of niacinamide, alpha-tocopheryl acetate, pyridoxine hydrochloride, thiamine hydrochloride, electrolytic iron, zinc, and the like.

Paragraph 46: The protein extrudate product of any one of Paragraphs 44-45, wherein the one or more coloring agent is a food grade coloring agent selected from the group consisting of carotenoids, betalains, curcumin, and the like.

Paragraph 47: The protein extrudate product of any one of Paragraphs 44-46, wherein the one or more flavoring agents is a food grade flavoring agent selected from the group consisting of natural flavoring (for example, natural vanilla flavor), artificial flavoring, fruit puree (e.g., dried blueberry puree, dried apple puree, etc.), and the like.

Paragraph 48: The protein extrudate product of any one of Paragraphs 44-47, wherein the one or more food grade additive is selected from the group consisting of mixed tocopherols, lecithin, dimodan, datem, and the like.

Paragraph 49: The protein extrudate product of any one of Paragraphs 33-48, further comprising one or more of: (a) micronutrient(s) at about 0.00004-0.0004 wt %, or at about 0.00005-0.0035 wt %, or at about 0.0005 wt %; (b) coloring agents at about 0.5-3 wt %, or at about 1-2 wt %, or at about 1.5 wt %; (c) flavoring agent(s) at about 0.5-3 wt %, or at about 1-2 wt %, or at about 1.5 wt %; and/or (d) food grade additive(s) at about 0.5-1.5 wt %, or at about 0.75-0.85 wt %, or at about 0.8 wt %.

Paragraph 50: The protein extrudate product of any one of Paragraphs 33-49, wherein the protein extrudate product is in the form of a puff food product.

Paragraph 51: The protein extrudate product of any one of Paragraphs 33-50, wherein the protein extrudate product is shaped in a geometric shape and size suitable for ease of consuming and/or handling.

Paragraph 52: The protein extrudate product of any one of Paragraphs 33-51, wherein the protein extrudate product is fast orally dissolving.

Paragraph 53: The protein extrudate product of any one of Paragraphs 33-52, wherein the protein extrudate product is protein-rich.

Paragraph 54: An edible food comprising the protein extrudate product of to any one of Paragraphs 33-53.

Paragraph 55: The edible food of Paragraph 54, wherein the edible food comprises a puff food product, a puffed and coated food product, and/or a food product of different shapes and sizes suitable for oral consumption.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, if provided, like reference numerals contained in the drawings are meant to identify similar or identical elements.

FIG. 1 is a flowchart of an embodiment of the process of preparation of quick disintegrating milk protein-based puffs according to the present disclosure.

FIG. 2 is a flowchart of a more particular embodiment of the process of preparation of quick disintegrating milk protein-based puffs according to the present disclosure.

FIG. 3 is a line diagram of an embodiment of an SCFX system used for the preparation of quick disintegrating milk protein-based puffs according to the present disclosure.

FIG. 4 is a photograph of quick disintegrating milk protein-based puffs according to an embodiment the present disclosure.

FIG. 5 is an FTIR absorbance spectra (950-1050 cm⁻¹) of MPC-S extrudates with different levels of SHMP.

FIG. 6 is an SDS-PAGE pattern of MPC extrudates prepared with different levels of SHMP. Lane 1 represents unextruded MPC. Lanes 2-5 represent 0 to 1% SHMP incorporation in MPC feeds. Lanes 6-10 represent the same samples in reducing conditions.

FIG. 7 is a graph illustrating Percent water uptake with time of MPC extrudates prepared with different levels of SHMP.

FIG. 8 is a graph illustrating the effect of hydration on compression strength of MPC extrudates prepared with different levels of SHMP.

FIG. 9 is a graph illustrating the effect of SC—CO₂ incorporation on protein extractability of extruded MPC.

FIG. 10 is a graph illustrating the effect of SC—CO₂ incorporation on free sulfhydryl content of extruded MPC.

FIG. 11 is an SDS-PAGE pattern of MPC extrudates prepared with different levels of SC—CO₂. Lanes 1-5 represent 0 to 0.9 kg/h SC—CO₂ input rate. Lanes 6-10 represent the same samples in reducing conditions.

FIG. 12A is a graph showing simulated pH and % CO₂ saturation in aqueous phase of the CO₂—H₂O system at 85° C. at different pressures.

FIG. 12B is a graph showing simulated pH and % CO₂ saturation in aqueous phase of the CO₂—H₂O and CO₂—H₂O-protein (60% MPC) system at 85° C. and 1600 psi (11.03 MPa).

FIG. 13 illustrates cellular morphology of milk protein concentrate puffs expanded by variable SC—CO₂ input rate at different pressures.

FIG. 14A is a graph showing percent extractable protein of unextruded, steam extruded and SCF extruded MPC80-S with different levels (0, 2.5 and 5 wt. % feed moisture) of SC—CO₂ injection.

FIG. 14B illustrates electrophoretic patterns of unextruded (lane 1 & 6), steam extruded (lane 2 & 7) and SCF extruded MPC80-S (SC-0: lane 3 & 8; SC-2.5: lane 4 & 9; SC-5: lane 5 & 10) in non-reducing (lanes 1-5) and reducing (β-mercaptoethanol) conditions (lanes 6-10).

FIGS. 15A-15D illustrates MPC extrudates prepared by SX and SCFX. FIG. 15A is steam extruded. FIG. 15B is SC-0 extruded. FIG. 15C is SC-2.5 extruded. FIG. 15D is SC-5 extruded.

FIG. 16 is a schematic of milk protein aggregation during steam (A) and SCF extrusion (B).

FIG. 17 is a graph showing simulated pH and % CO₂ saturation in aqueous phase of the CO₂—H₂O and CO₂—H₂O-protein (60% MPC) system at 85° C. and 1600 psi (11.03 MPa).

FIG. 18 illustrates electrophoretic patterns of unextruded (lane 1 & 6), steam extruded (lane 2 & 7) and SCF extruded MPC80-S (lane 4 & 9), and steam extruded (lane 3 & 8) and SCF extruded MPC80-S-SHMP (lane 4 & 9) in non-reducing (lanes 1-5) and reducing (β-mercaptoethanol) conditions (lanes 6-10).

FIG. 19 is a graph showing percent water uptake with time of MPC extrudates prepared with and without added SHMP.

FIG. 20 is a graph showing the effect of hydration on compression strength of extrudates prepared with and without added SHMP and commercial sample.

FIG. 21 is a graph showing textural analysis (two bite test—with hydration) of MPC-SHMP and commercial sample.

FIG. 22 is a schematic of protein aggregation illustrating the effect of incorporation of SHMP in MPC extrudates prepared by SCFX vs SX.

FIG. 23 is a table showing the mass, shape and dimensions of the puffed food samples of the present disclosure provided to the panelists for evaluation of disintegration time and other sensory attributes.

FIGS. 24A-24B are graphs showing the demographics of the panelists selected for sensory evaluation of products to be potentially used as baby puffs. FIG. 24A shows the demographics by gender. FIG. 24B shows the demographics according to the panelists' familiarity with the product category.

FIGS. 25A-25C are graphs showing the mean in-mouth disintegration time (FIG. 25A), disintegration profile-mean (FIG. 25B), and disintegration profile (median) (FIG. 25C) of the puffed products evaluated by sensory evaluation panel.

FIGS. 26A-26D are graphs showing just about right (JAR) scales for roughness (FIG. 26A), sweetness (FIG. 26B), disintegration time (FIG. 26C), and presence of residue after disintegration (FIG. 26D) of the puffed products evaluated by sensory evaluation panel.

### DETAILED DESCRIPTION

The present disclosure provides, inter alia, methods of preparing an orally dissolving protein extrudate product, orally dissolving protein extrudate products, and edible foods containing the orally dissolving protein extrudate products. These aspects of the present disclosure are further described herein.

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise.

The antecedent “about” indicates that the values are approximate. For example the range of “about 1% to about 50% by weight” indicates that the values are approximate values. The range of “about 1% to about 50% by weight” includes approximate and specific values, e.g., the range includes about 1%, 1%, about 50% and 50%.

When a range is described, the range includes both the endpoints of the range as well as all numbers in between. For example, “between 1% and 10%” includes 1%, 10% and all amounts between 1% and 10%. Likewise, “from 1% to 10%” includes 1%, 10% and all amounts between 1% and 10%.

As used herein, the term “orally dissolving” refers to an edible composition's ability to dissolve or disintegrate in the oral cavity of a human either with or without the addition of an extraneous hydration source (e.g., water). As used herein, in particular examples, the term “orally dissolving” refers to an edible composition's ability to dissolve or disintegrate in-mouth due to saliva as the only or primary source of hydration. The term “orally dissolving” can be used interchangeably with the term “orally disintegrating” or other similar terms. Examples of “orally dissolving” edible compositions include the protein extrudate products and edible foods described herein.

As used herein, the term “fast orally dissolving” refers to an edible composition's ability to dissolve or disintegrate in the oral cavity of a human in 30 seconds or less, either with or without the addition of an extraneous hydration source (e.g., water). As used herein, in particular examples, the term “fast orally dissolving” refers to an edible composition's ability to dissolve or disintegrate in-mouth in 30 seconds or less due to saliva as the only or primary source of hydration. The term “fast orally dissolving” can be used interchangeably with the term “fast orally disintegrating,” “rapid orally dissolving,” “rapid orally disintegrating,” “quick orally dissolving,” “quick orally disintegrating,” or other similar terms. Examples of “fast orally dissolving” edible compositions include the protein extrudate products and edible foods described herein.

As used herein, the term “protein-rich” refers an edible composition having a protein content of about 65% or greater. As used herein, examples of protein-rich edible compositions include, without limitation, the protein extrudate products described herein and the edible foods containing or made of the protein extrudate products having proteins in an amount of about 65% or greater. As used herein, “protein-rich” can refer to edible compositions having a protein content of about 70% or greater, about 75% or greater, about 80% or greater, about 85% or greater, about 90% or greater, or about 95% or greater. Percentage of protein content can be determined using calculation methods known in the art. One method can include calculating the weight percent (wt %) of the protein in the protein extrudate product and other edible foods of the present disclosure.

### Methods of Preparing Orally Dissolving Protein Extrudate Products

In one aspect, the present disclosure provides methods of preparing orally dissolving protein extrudate products, orally dissolving protein extrudate products that are protein-rich, fast orally dissolving protein extrudate products, and fast orally dissolving protein extrudate products that are protein-rich.

In accordance with the present disclosure, the method of preparing orally dissolving protein extrudate products includes the steps of: (a) blending a protein concentrate with one or more extrusion component to produce a protein dough; (b) extruding the protein dough with supercritical fluid to form a wet protein extrudate, the extruding being effective to inhibit protein-protein interactions in the protein concentrate; and (c) drying the wet protein extrudate to form a protein extrudate product that is orally dissolving. In certain embodiments, the method is effective to prepare fast orally dissolving protein extrudate products.

In various embodiments of this method, the protein concentrate is milk protein concentrate (MPC). In various other embodiments, the protein concentrate can be from other animal protein sources (e.g., yogurt concentrate or powder) and from other sources. In certain embodiments, the protein concentrate is prepared by diafiltration using techniques known in the food processing field.

Milk protein concentrates (MPC) are manufactured using pressure-driven membrane separation processes, where ultrafiltration (UF) alone, or a combined UF and diafiltration (DF) process (depending upon the final protein concentration required), is used to concentrate protein while removing smaller molecules including lactose, salts, and non-protein nitrogen followed by spray drying (Carr & Golding, 2016). In certain embodiments, the term MPC refers to milk protein concentrates with more than 80% protein.

As casein proteins are rich in non-polar, hydrophobic amino acids, non-covalent hydrophobic interactions are formed during spray drying resulting in poor solubility of MPC. Hydrophobic interactions prevent the breakdown of agglomerated particles. Therefore, the use of an MPC-based method of the present disclosure enables the production of quick disintegrating, easy-to-swallow puffs, which involve the minimization of these interactions during extrusion.

Therefore, in accordance with the present disclosure, the disclosed method is effective to minimize the protein-protein interactions during extrusion, where the extrusion conditions are such that they minimize the calcium bridging and hydrophobic interactions amongst caseins and also prevent casein-whey protein and whey protein-whey protein aggregation.

In various embodiments of this method, the one or more extrusion component can include, without limitation, one or more sugar, emulsifier, and/or dispersing agent.

As used herein, “sugar” can include one or more saccharide, including monosaccharides, disaccharides, and/or oligosaccharides. In certain embodiments, the monosaccharides can include, without limitation, glucose and/or fructose. In certain embodiments, the disaccharides can include, without limitation, sucrose and/or maltose. In certain embodiments, the oligosaccharides can include, without limitation, maltodextrins. In a particular embodiment, the saccharide is sucrose.

As used herein, “emulsifier” can include any food grade emulsifier. Suitable food grade emulsifiers can include, without limitation, lecithin, distilled monoglycerides, polysorbates, diacetyl tartaric esters of mono and diglycerides, and the like. In certain embodiments, more than one emulsifier is used in the extrusion component during execution of the blending step of the disclosed method.

As used herein, “dispersing agent” can include any chelator effective for chelating calcium. Suitable dispersing agents can include, without limitation, sodium hexametaphosphate (SHMP), trisodium diphosphate, tetrasodium diphosphate, and the like. In a particular embodiment, the dispersing agent is SHMP.

Sodium hexametaphosphate is a food additive (E452i) used in a variety of food products as a chelator, sequestrant, thickener, emulsifier, and texturizer. SHMP has a wide range of uses in the food industry such as increasing the water binding properties of proteins in processed meats, protein precipitation for purification purposes, and prevention of protein sedimentation in sterilized milk (Molins, 1991). SHMP chelates the calcium ions from casein micelles resulting in their dissociation, which has shown to improve to solubility and heat stability of milk proteins.

In accordance with the present disclosure, utilization of SHMP as a functional ingredient at low processing temperatures prevents casein aggregation and temporarily imposed acidic conditions due to the incorporation of SC—CO₂ during extrusion can minimize calcium bridging and the formation of disulfide linkages of casein-whey protein and whey protein-whey protein type. Thus, in certain embodiments, the method of the present disclosure employs the low-temperature and temporarily-imposed acidity to MPC incorporated with selected diluents and chelating agents via high-pressure, carbon dioxide-based high-moisture extrusion to develop extrudates with defined microstructure facilitating quick disintegration.

Thus, in certain embodiments, the use of SHMP in the feed formula in tandem with the temporary lowered pH during supercritical fluid extrusion using carbon dioxide is advantageous to yield the desired orally dissolving protein-rich extrudates of the present disclosure.

As used in the methods disclosed herein, the protein dough can include the various disclosed ingredients in various amounts, with the disclosed ingredients of the protein dough at least including the protein concentrate, sugar(s), emulsifier(s), and dispersing agent(s). In other embodiments, the protein dough also includes water. In other embodiments, the protein dough can further include other optional ingredients, such as any other edible constituent that is suitable for use for human consumption and that does not inhibit or prevent the finished protein extrudate product or food product thereof from being orally dissolving, fast orally dissolving, orally dissolving and protein-rich, or fast dissolving and protein-rich.

In certain embodiments, the protein dough includes protein concentrate at about 40-80 wt %, about 45-75 wt %, about 50-70 wt %, about 55-65 wt %, about 57-64 wt %, about 58-63 wt %, about 59-62 wt %, or about 60-61 wt %. In a particular embodiment, the protein concentrate is included in the protein dough at about 60 wt %.

In certain embodiments, the protein dough includes sugar at about 2-20 wt %, about 4-18 wt %, about 5-15 wt %, about 6-12 wt %, about 8-11 wt %, about 9-10 wt %. In a particular embodiment, the sugar is included in the protein dough at about 9.6 wt %.

In certain embodiments, the protein dough includes emulsifier(s) at about 0-3 wt %, about 0.1-2.8 wt %, about 0.2-2.6 wt %, about 0.3-2.4 wt %, about 0.4-2.2 wt %, about 0.5-2.0 wt %, about 0.6-1.8 wt %, about 0.7-1.6 wt %, about 0.8-1.8 wt %, about 1.0-1.6 wt %, or about 1.2-1.4 wt %. In a particular embodiment, the emulsifier(s) are included in the protein dough at about 1.42 wt %.

In certain embodiments, the protein dough includes dispersing agent(s) at about 0.1-0.5 wt %, about 0.15-0.5 wt %, about 0.2-0.5 wt %, about 0.25-0.5 wt %, about 0.3-0.5 wt %, about 0.35-0.5 wt %, about 0.4-0.5 wt %, about 0.45-0.5 wt %, or about 0.3-0.4 wt %. In a particular embodiment, the dispersing agent(s) are included in the protein dough at about 0.357 wt %.

In certain embodiments, the protein dough includes water at about 20-40 wt %, at about 25-35 wt %, about 26-33 wt %, about 27-32 wt %, about 28-31 wt %, about 28-30 wt %, or about 28-29 wt %. In a particular embodiment, water is included in the protein dough at about 28.5 wt %.

In certain embodiments, the protein dough includes: (a) protein concentrate at about 40-80 wt %, or at about 50-70 wt %, or at about 60 wt %; (b) sugar at about 2-20 wt %, or at about 5-15 wt %, or at about 9.6 wt %; (c) emulsifier(s) at about 0-3 wt %, or at about 0.5-2 wt %, or at about 1.42 wt %; (d) dispersing agent at about 0.1-0.5 wt %, or at about 0.3-0.5 wt %, or at about 0.357 wt %; and (e) water at about 20-40 wt %, or at about 25-35 wt %, or at about 28.5 wt %.

In certain embodiments, the protein dough includes: (a) protein concentrate at about 40-80 wt %; (b) sugar at about 2-20 wt %; (c) emulsifier(s) at about 0-3 wt %; (d) dispersing agent at about 0.1-0.5 wt %; and (e) water at about 20-40 wt %.

In certain embodiments, the protein dough includes: (a) protein concentrate at about 50-70 wt %; (b) sugar at about 5-15 wt %; (c) emulsifier(s) at about 0.5-2 wt %; (d) dispersing agent at about 0.3-0.5 wt %; and (e) water at about 25-35 wt %.

In a particular embodiment, the protein dough includes: (a) protein concentrate at about at about 60 wt %; (b) sugar at about 9.6 wt %; (c) emulsifier(s) at about 1.42 wt %; (d) dispersing agent at about 0.357 wt %; and (e) water at about 28.5 wt %.

In a particular embodiment, the protein dough includes about 840 kg of protein concentrate, about 135 kg of saccharide, about 10 kg of lecithin, about 10 kg of distilled monoglycerides, and about 5 kg of dispersing agents.

During the blending step of the disclosed method, various procedures known in the food manufacturing art can be used to blend the various disclosed ingredients according to the present disclosure to yield the protein dough prior to the extruding step. As described herein, such disclosed ingredients of the protein dough include at least the protein concentrate, sugar(s), emulsifier(s), and dispersing agent(s), and can also include water, and optionally other optional ingredients, such as any other edible constituent that is suitable for use for human consumption and that does not inhibit or prevent the finished protein extrudate product or food product thereof from being orally dissolving, fast orally dissolving, orally dissolving and protein-rich, or fast dissolving and protein-rich.

In accordance with the disclosed method, certain other additional ingredients may be added during the various steps of the method according to practices known in the food manufacturing art. In certain embodiments, the additional ingredients can include, without limitation, micronutrients, coloring agents, flavoring agents, and any other food grade additives. In certain embodiments, the micronutrients, coloring agent(s), flavoring agent(s), and/or other food grade additives can be added in the feed formula prior to extrusion or can be sprayed on and/or coated upon the end product after extrusion.

As used herein, “micronutrients” can include, without limitation, niacinamide, alpha-tocopheryl acetate, pyridoxine hydrochloride, thiamine hydrochloride, electrolytic iron, zinc, and the like. In certain embodiments, the micronutrient(s) can be added during the method at about 0.00004-0.0004 wt %, or at about 0.00005-0.0035 wt %, or at about 0.0005 wt %.

As used herein, “coloring agents” can include, without limitation, any food grade coloring agent. In certain embodiments, the micronutrients can include, without limitation, carotenoids, betalains, curcumin, and the like. In certain embodiments, the coloring agent(s) can be added during the method at about 0.5-3 wt %, or at about 1-2 wt %, or at about 1.4 wt %.

As used herein, “flavoring agents” can include, without limitation, any food grade flavoring agent. In certain embodiments, the flavoring agent can include, without limitation, natural flavoring, artificial flavoring, fruit puree, and the like of any flavor suitable for use in food preparation. In certain embodiments, the natural flavoring can include, for example, natural vanilla flavor and the like. In certain embodiments, the fruit puree can include, for example, dried blueberry puree, dried apple puree, and the like. In certain embodiments, the flavoring agent(s) can be added during the method at about 0.5-3 wt %, or at about 1-2 wt %, or at about 1.4 wt %.

As used herein, “food grade additives” can include any other additive suitable for use in food preparation. In certain embodiments, the food grade additives can include, without limitation, tocopherols, lecithin, dimodan, datem, and the like. In certain embodiments, the food grade additive(s) can be added during the method at about at about 0.5-1.5 wt %, or at about 0.75-1.0 wt %, or at about 0.85 wt %.

In certain embodiments, the blending step involves dry blending. In a particular embodiment, the dry blending involves, without limitation, blending in a ribbon blender for about 5-30 minutes, about 10-25 minutes, about 15-20 minutes, or for about 10, 15, 20, 25, or 30 minutes.

During the extruding step of the disclosed method, the protein dough is extruded with a supercritical fluid. In one embodiment, the supercritical fluid is carbon dioxide.

Supercritical fluid extrusion (SCFX) technology combines the plasticizing and expanding properties of SC—CO₂ with conventional extrusion to generate microcellular foam of feedstock at low temperatures (<100° C.) (Rizvi and Mulvaney, 1992). Suitable process variables and formulation on expansion, cell size, cell density, and mechanical properties of SC—CO₂ injection-based extrudates previously described have been modified in accordance with the present disclosure (Cho and Rizvi, 2009 a & b). In accordance with the present disclosure, extension of this benign extrusion technology, which retains the nutritional and organoleptic qualities, offers attractive options for the manufacture of novel dairy-based products such as those using MPC as a base. Thus, extrusion processing as disclosed herein can be employed to modify the properties of MPC and prepare extruded products with altered physico-functional characteristics.

Suitable examples of the use of supercritical fluid extrusion can be found in U.S. Pat. No. 10,524,497 to Rizvi et al. (referred to herein as “Rizvi '497”), which is incorporate herein by reference. As discussed in Rizvi '497, supercritical fluids have desirable properties such as gas-like diffusivity and viscosity and liquid-like density and are utilized in a variety of food and industrial applications. Carbon dioxide (CO₂) is the most common supercritical fluid and is regarded as an inert, non-toxic, naturally abundant, tunable, and non-flammable solvent with relatively low critical pressure (7.38 MPa) and temperature (31.1° C.). Supercritical fluid extrusion (SCFX) combines extrusion processing with supercritical fluids to overcome the limitations of the conventional high temperature steam extrusion. This is achieved by incorporating supercritical CO₂ as a blowing agent instead of steam. The SCFX process produces low density expanded products at low-temperature and -shear conditions, which allows incorporating heat and shear sensitive ingredients such as proteins and micronutrients in extruded products.

The key parameters or conditions of the SCFX process as used in the disclosed method include feed rate, screw speed, pressure, and temperature. These various parameters (e.g., feed rate, screw speed, pressure, and temperature) of the SCFX conditions are as described generally and in more detail in the present disclosure. Certain embodiments of these parameters are described herein below.

Feed Rate: As provided herein, the protein dough is extruded with the supercritical fluid at a feed rate of about 1-50 kg/h, about 5-45 kg/h, about 10-45 kg/h, about 20-45 kg/h, about 25-40 kg/h, about 30-40 kg/h, about 31-39 kg/h, about 32-38 kg/h, about 33-37 kg/h, or about 34-36 kg/h. In a particular embodiment, the protein dough is extruded with the supercritical fluid at a feed rate of about 35 kg/h. In a particular embodiment, the protein dough is extruded with the supercritical fluid at a supercritical fluid feed rate of about 0.8 kg/h. In a particular embodiment, the protein dough is extruded with the supercritical fluid at a water feed rate of about 14 kg/h.

Screw Speed: As provided herein, the protein dough is extruded with the supercritical fluid at a screw speed of about 1-200 rpm, about 10-190 rpm, about 20-180 rpm, about 30-170 rpm, about 40-160 rpm, about 50-150 rpm, about 60-140 rpm, about 70-130 rpm, about 80-120 rpm, about 90-118 rpm, about 100-116 rpm, about 103-114 rpm, about 106-112 rpm, or about 109-111 rpm. In a particular embodiment, the protein dough is extruded with the supercritical fluid at a screw speed of about 110 rpm.

Pressure: As provided herein, the protein dough is extruded with the supercritical fluid at a supercritical fluid injection pressure of about 1 to 20 MPa, about 2 to 19 MPa, about 3 to 18 MPa, about 4 to 17 MPa, about 5 to 16 MPa, about 6 to 15 MPa, about 7 to 14 MPa, about 8 to 13 MPa, about 9 to 12 MPa, or about 10 to 12 MPa. In a particular embodiment, the protein dough is extruded with the supercritical fluid at a supercritical fluid injection pressure of about 11 MPa.

Temperature: As provided herein, supercritical extrusion may be conducted at temperatures of less than 100° C., less than 95° C., less than 90° C., less than 85° C., less than 80° C., less than 75° C., less than 70° C., less than 65° C., less than 60° C., less than 55° C., less than 50° C., less than 45° C., or less than 40° C. In certain embodiments, the protein dough is extruded with the supercritical fluid at a barrel set temperature of about 5-40° C., about 10-40° C., about 15-40° C., about 20-40° C., about 22-40° C., about 24-40° C., about 26-40° C., about 28-40° C., about 30-40° C., about 32-40° C., about 34-40° C., about 36-40° C., or about 38-40° C.

In certain embodiments, in accordance with the extruding step of the disclosed method, the wet protein extrudate is collected at a temperature of between about 70-95° C., about 75-90° C., or about 80-85° C. In certain other embodiments, the wet protein extrudate is collected at a temperature of about 70° C., about 75° C., about 80° C., about 85° C., about 90° C., or about 95° C. In a particular embodiment, the wet protein extrudate is collected at a temperature of about 85° C.

During the drying step of the disclosed method, various drying procedures know in the food manufacturing art may be used to dry the wet protein extrudate to form the protein extrudate product that is orally dissolving, fast orally dissolving, orally dissolving and protein-rich, or fast dissolving and protein-rich.

In certain embodiments, in accordance with the drying step of the disclosed method, the drying can include, without limitation, conventional oven drying, vacuum oven drying, or the like.

In certain embodiments, in accordance with the drying step of the disclosed method, the drying can include, without limitation, drying until a moisture content of the protein extrudate product is less than about 5-15%. In particular embodiments, the drying step is conducted until a moisture content of the protein extrudate product is at least less than about 5%, less than about 10%, or less than about 15%. Suitable techniques for measuring the temperature of the protein extrudate product during the drying step are those that are known in the relevant art.

In certain embodiments, in accordance with the drying step of the disclosed method, the drying can include, without limitation, conventional drying at temperatures of greater than about 50-95° C. with or without vacuum. In particular embodiments, the conventional drying can be at temperatures of greater than about 50° C., 55° C., 60° C., 65° C., 70° C., 75° C., 80° C., 85° C., 90° C., or 95° C., with or without vacuum.

### Orally Dissolving Protein Extrudate Products and Edible Foods

In another aspect, the present disclosure provides orally dissolving protein extrudate products. In certain embodiments, the orally dissolving protein extrudate products are protein-rich, fast orally dissolving protein extrudate products, or fast orally dissolving protein extrudate products that are also protein-rich. The orally dissolving protein extrudate products can be prepared according to the methods described herein.

As disclosed herein, the orally dissolving protein extrudate products of the present disclosure include the following ingredients: protein concentrate, sugar(s), emulsifier(s), and dispersing agent(s), which ingredients are as disclosed elsewhere herein and/or below.

In various embodiments, protein concentrate in the orally dissolving protein extrudate includes, without limitation, a milk protein concentrate (MPC). In various other embodiments, the protein concentrate can be from other animal protein sources (e.g., yogurt concentrate or powder) and from other sources. In certain embodiments, the protein concentrate is prepared by ultrafiltration using techniques known in the food processing field. In certain embodiments, the protein concentrate is prepared by diafiltration using techniques known in the food processing field.

In various embodiments, the sugar in the orally dissolving protein extrudate includes, without limitation, one or more saccharide, including monosaccharides, disaccharides, and/or oligosaccharides. In certain embodiments, the monosaccharides can include, without limitation, glucose and/or fructose. In certain embodiments, the disaccharides can include, without limitation, sucrose and/or maltose. In certain embodiments, the oligosaccharides can include, without limitation, maltodextrins. In a particular embodiment, the saccharide is sucrose.

In various embodiments, the emulsifier can include, without limitation, any food grade emulsifier. Suitable food grade emulsifiers can include, without limitation, lecithin, distilled monoglycerides, polysorbates, diacetyl tartaric esters of mono and diglycerides, and the like. In certain embodiments, more than one emulsifier is included in the orally dissolving protein extrudate.

In various embodiments, the dispersing agent can include, without limitation, any chelator effective for chelating calcium. Suitable dispersing agents can include, without limitation, sodium hexametaphosphate (SHMP), trisodium diphosphate, tetrasodium diphosphate, and the like. In a particular embodiment, the dispersing agent is SHMP.

The orally dissolving protein extrudate products of the present disclosure can also include, without limitation, water and other ingredients, such as micronutrients, coloring agents, flavoring agents, and/or other food grade additives. Additional ingredients may be added in a combination and/or in an amount so that the final protein extrudate product or edible food either maintains or does not maintain the orally dissolving or fast orally dissolving characteristic of the protein extrudate product itself. According to certain embodiments of the present disclosure, the orally dissolving, fast orally dissolving, and/or protein-rich characteristic of the protein extrudate product of the present disclosure remains intact, even if adding additional ingredients to arrive at a final food product would make that final food product not completely orally dissolving, fast orally dissolving, and/or protein-rich.

In certain embodiments, the protein extrudate product includes: (a) a protein concentrate; (b) a sugar; (c) an emulsifier; and (d) a dispersing agent comprising a calcium chelating agent, where the protein extrudate product is orally dissolving and protein-rich, fast orally dissolving, or fast orally dissolving and protein-rich.

In certain embodiments, the protein extrudate product includes: (a) protein concentrate at about 60-90 wt %, or at about 65-85 wt %, or at about 84 wt %; (b) sugar at about 5-25 wt %, or at about 10-20 wt %, or at about 13.5 wt %; (c) emulsifier(s) at about 0.05-4 wt %, or at about 1-3 wt %, or at about 1-2 wt %; and (d) dispersing agent at about 0.1-0.5 wt %, or at about 0.3-0.5 wt %, or at about 0.4-0.5 wt %.

In certain embodiments, the protein extrudate product includes: about 84 wt % milk protein concentrate, about 13.5 wt % sucrose, about 1 wt % distilled monoglycerides, and about 0.5 wt % sodium hexametaphosphate.

In certain embodiments, the protein extrudate product includes: (a) protein concentrate at about 60-90 wt %, or at about 65-85 wt %, or at about 84 wt %; (b) sugar at about 5-25 wt %, or at about 10-20 wt %, or at about 13.5 wt %; (c) emulsifier(s) at about 0.05-4 wt %, or at about 1-3 wt %, or at about 1-2 wt %; and (d) dispersing agent at about 0.1-0.5 wt %, or at about 0.3-0.5 wt %, or at about 0.4-0.5 wt %.

In certain embodiments, the protein extrudate product has a stable shelf life. In certain embodiments, the protein extrudate product has a shelf life of more than about 3-12 months, or more than about 3, 6, 9, or 12 months. In certain embodiments, the protein extrudate product is a dry product. Suitable examples of the dry product of the present disclosure includes a protein extrudate product having a moisture content of less than about 5-15%. In particular embodiments, the moisture content of the protein extrudate product is at least less than about 5%, less than about 10%, or less than about 15%.

In certain embodiments, the protein extrudate product can include water and/or other optional ingredients, such as any other edible constituent that is suitable for use for human consumption and that does not inhibit or prevent the finished protein extrudate product or food product thereof from being orally dissolving, fast orally dissolving, orally dissolving and protein-rich, or fast dissolving and protein-rich.

In certain embodiments, the other additional ingredients may be added according to practices known in the food manufacturing art. In certain embodiments, the additional ingredients can include, without limitation, micronutrients, coloring agents, flavoring agents, and any other food grade additives.

In certain embodiments, the micronutrients can include, without limitation, niacinamide, alpha-tocopheryl acetate, pyridoxine hydrochloride, thiamine hydrochloride, electrolytic iron, zinc, and the like. In certain embodiments, the micronutrient(s) can be added during the method at about 0.00004-0.0004 wt %, or at about 0.00005-0.0035 wt %, or at about 0.0005 wt %.

In certain embodiments, the coloring agents can include, without limitation, any food grade coloring agent. In certain embodiments, the micronutrients can include, without limitation, carotenoids, betalains, curcumin, and the like. In certain embodiments, the coloring agent(s) can be added during the method at about 0.5-3 wt %, or at about 1-2 wt %, or at about 1.5 wt %.

In certain embodiments, the flavoring agents can include, without limitation, any food grade flavoring agent. In certain embodiments, the flavoring agent can include, without limitation, natural flavoring, artificial flavoring, fruit puree, and the like of any flavor suitable for use in food preparation. In certain embodiments, the natural flavoring can include, for example, natural vanilla flavor and the like. In certain embodiments, the fruit puree can include, for example, dried blueberry puree, dried apple puree, and the like. In certain embodiments, the flavoring agent(s) can be added during the method at about 0.5-3 wt %, or at about 1-2 wt %, or at about 1.5 wt %.

In certain embodiments, the food grade additives can include any other additive suitable for use in food preparation. In certain embodiments, the food grade additives can include, without limitation, tocopherols, lecithin, dimodan, datem, and the like. In certain embodiments, the food grade additive(s) can be added during the method at about at about 0.5-1.5 wt %, or at about 0.75-0.85 wt %, or at about 0.8 wt %.

In certain embodiments, the protein extrudate product is in the form of a puff food product. Suitable examples of the protein extrudate product can include, without limitation, milk protein puffs with quick in-mouth dissolving characteristics suitable for consumption by toddlers, the elderly, and dysphagic patients.

In certain embodiments, the protein extrudate product is shaped in various geometric shapes and/or sizes suitable for ease of consuming and/or handling.

In certain embodiments, the final product can be coated or co-extruded with other edible items, including, without limitation, chocolate and other coatings, fillings, or the like.

In another aspect, the present disclosure provides edible foods containing or made of the protein extrudate products disclosed herein. The edible foods can include, without limitation, any food for human consumption that can include the orally dissolving protein extrudate product of the present disclosure. Suitable examples of edible foods can include, without limitation, the use of the protein extrudate product of the present disclosure as components of other foods, including, for example, nutrition bars, protein bars, fruit bars, granola bars, snack foods, breakfast cereals, dessert toppings, salad toppings, other food toppings, trail mixes, and the like. In certain embodiments, the protein extrudate product of the present disclosure is in the form of a puff and used as a component with another food to produce a final edible food product. Suitable food component combination techniques known in the food processing field can be used to produce such final edible food products.

In certain embodiments, the edible food can include, without limitation, a puff food product, a puffed and coated food product, and/or a food product of different shapes and sizes suitable for oral consumption.

Without being bound by theory, utilization of supercritical carbon dioxide during extrusion of MPC (Milk Protein Concentrate) may prevent sulfhydryl interactions between whey proteins and k-casein owing to the pH reducing effect of carbonic acid. Incorporation of SHMP (Sodium hexametaphosphate) in MPC feed may chelate calcium and prevent the calcium bridging between proteins during extrusion. Reduction of the above-mentioned interactions may lead to weakening of the solid bridges that make the expanded structure of the extrudate, resulting in extruded product that may collapse easily on hydration (or in-mouth due to saliva).

In certain embodiments, the method of the present disclosure utilizes supercritical fluid extrusion (SCFX) technology to manufacture milk protein-based, in-mouth disintegrating puffs formulated with milk protein concentrate with added sugar (sucrose), emulsifiers (lecithin and distilled monoglycerides) and a dispersing agent sodium hexametaphosphate (SHMP). These puffs are intended to be used as protein-rich, nutritionally superior yet convenient baby foods. The fast disintegrating milk protein puffs of the present disclosure are a valuable replacement for starch-based first finger foods currently available in the market for toddlers. The milk protein puffs of the present disclosure may also be equally attractive and useful for elderly and dysphagia patients or patients with other conditions that make chewing and swallowing difficult.

The SCFX process of the present disclosure can manufacture milk protein-based puffs with quick, in-mouth disintegrating characteristics that offer many advantages over currently existing baby foods. Currently available products either offer protein-rich puffed products for early childhood (3-8 years old) or protein-rich pureed products for infants and toddlers (6 months-2 years old). It is understood that no protein-rich puffed products suitable for infants and toddlers have been reported.

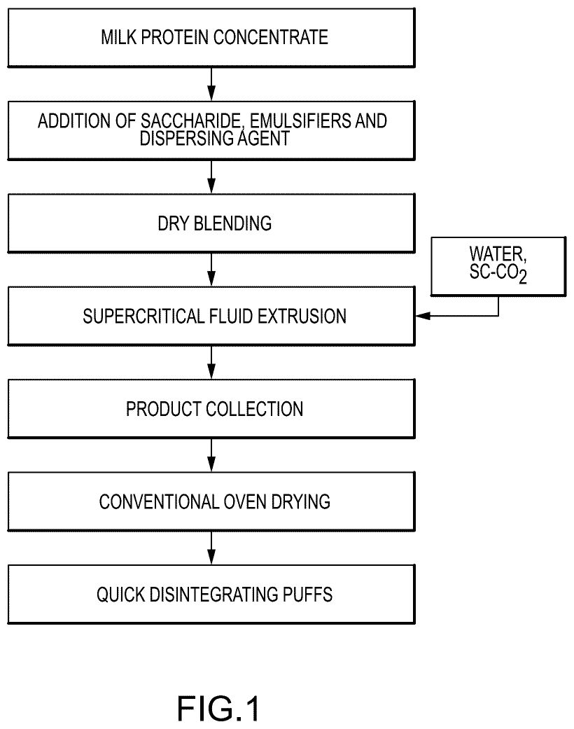

As provided herein, in certain embodiments, the presently disclosed method uses the SCFX technology to manufacture milk protein-based, in-mouth disintegrating puffs formulated with a saccharide (sucrose), emulsifiers (lecithin and distilled monoglycerides), and dispersing agent (SHMP). The milk protein-based puffs are intended to be used as nutritionally superior yet convenient baby foods. A flow chart of one embodiment of a process of preparation of quick disintegrating milk protein-based puffs is found in FIG. 1.

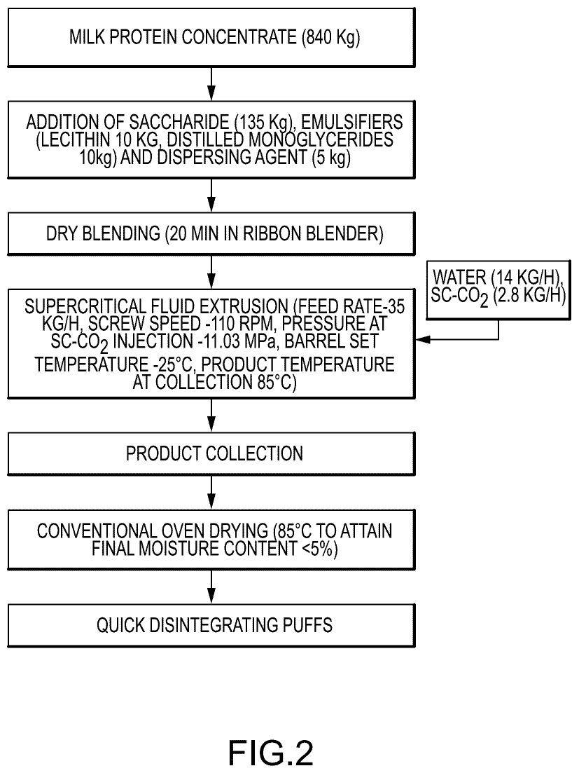

An exemplary method of the present disclosure is shown in FIG. 2, as discussed below. The dry ingredients (weight percent formulation: MPC—84%, sucrose—13.5%, lecithin—1%, distilled monoglycerides—1%, SHMP—0.5%) were blended in a ribbon blender and stored until extrusion. The formulations were extruded using a co-rotating, twin-screw extruder (Wenger TX-52 Magnum, Sabetha KS, USA) coupled with supercritical carbon dioxide (SC—CO₂) injection system. The extruder with an L/D ratio of 28.5:1 is configured for SC—CO₂ injection into the barrel at an L/D ratio of 24. The extruder was fitted with die plate having two circular die openings of 4.4 mm diameter each. The extruder was operated at a screw speed of 110 rpm at a feed rate of 35 kg/h. The extruder barrel zone temperatures were set at 25° C. The extruder was operated at 110 rpm at a feed rate of 35 kg/h. SC—CO₂ (2.8-3% dry feed) was continuously injected at a pressure of 1600 psi (11.03 MPa). The water input rate was maintained at 14 kg/h. The product temperature while collection at the die exit was 85° C. The extrudates were dried in the hot air convection oven at 80° C. for approximately 2-3 h to achieve a final moisture content of less than 5% (dry basis). The dried extrudates were packaged in airtight bags/plastic containers. As noted, a flow chart of one embodiment of an optimized process of preparation of quick disintegrating milk protein-based puffs is found in FIG. 2.

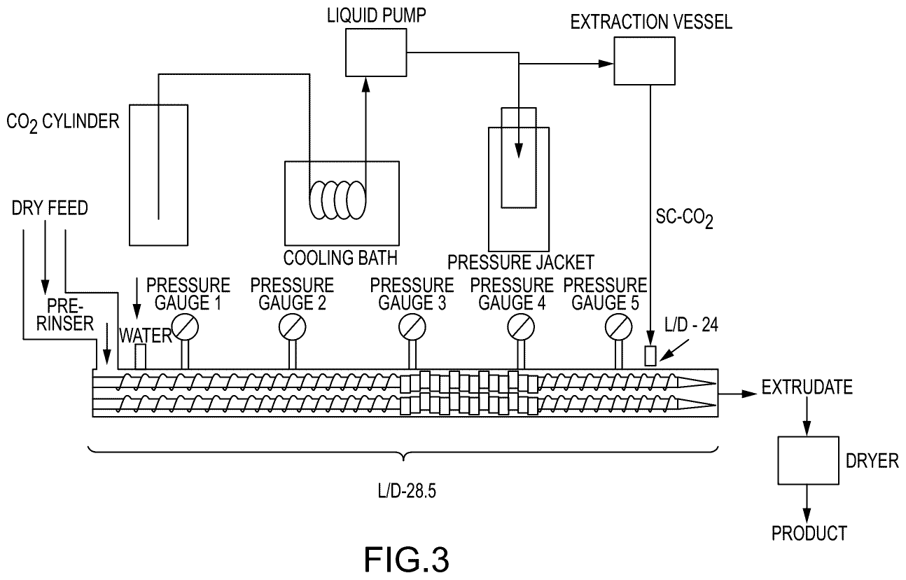

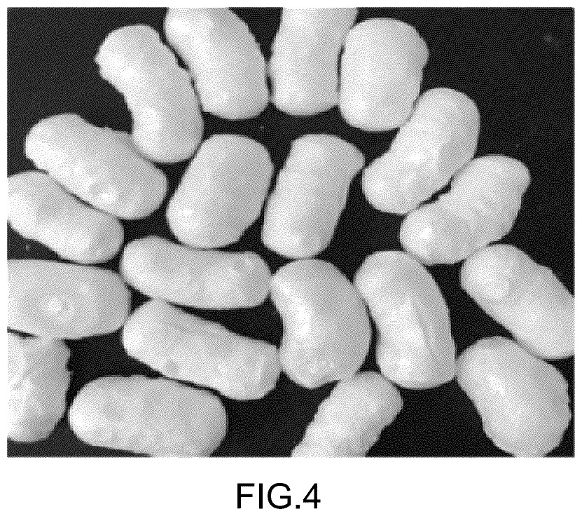

Examples of aspects of the systems and processes of the present disclosure are illustrated in FIG. 3 and FIG. 4. FIG. 3 is a line diagram of an embodiment of an SCFX system of the present disclosure used for the preparation of quick disintegrating milk protein-based puffs. FIG. 4 show examples of quick disintegrating milk protein-based puffs of the present disclosure.

Possible alternative versions of the method of the present disclosure can include, but are not limited to:
Replacing sucrose with other soluble saccharides (mono or dimeric) and SHMP with other polyphosphate salts or calcium chelating agents;
Adding a cocktail of micronutrients, color and flavor as needed;
Extrudates may be shaped in different geometries and sizes for ease of eating/handling;
High moisture, low temperature extrusion, using other forms of blowing agents, e.g., sodium bicarbonate, as a means to puff;
Milk protein concentrate of different protein content, as well as other plant and animal proteins, may be used as major ingredients in the formulation;
Coating or co-extruding the final product with other edible items, e.g., chocolate and others.

Advantages and/or uses of the orally dissolving protein extrudate products of the present disclosure include, but are not limited to:
Quick disintegrating milk protein puffs are useful as a valuable replacement for starch-based first finger foods currently in the market and intended for toddlers. They may offer the convenience of self-feeding by the child, portability and mess-free eating environment along with being rich in protein and provide essential amino acids through diet.
Designed with an adequate micronutrient profile, such products will be also useful in places where shelf-life and water quality may be an issue.
They provide opportunity for baby-led weaning instead of conventional parent dependent spoon-feeding. The infants and toddlers can participate in family mealtimes and they can feed themselves from the beginning of complementary feeding, making the child an active partner in the feeding process and assume healthy eating practices.
The milk protein puffs may also be useful for elderly and dysphagia patients or patients with other conditions that make chewing and swallowing difficult.

### EXAMPLES

The following examples are intended to illustrate particular embodiments of the present disclosure, but are by no means intended to limit the scope of the present disclosure.

### Example 1

### Orally Dissolving Milk Protein Puffs by Reactive Supercritical Fluid Extrusion

### Hypotheses

Utilization of supercritical carbon dioxide during extrusion of MPC (Milk Protein Concentrate) will prevent sulfhydryl interactions between whey proteins and k-casein owing to the pH reducing effect of carbonic acid.

Incorporation of SHMP (Sodium hexametaphosphate) in MPC feed will chelate calcium and prevent the calcium bridging between proteins during extrusion.

Reduction of the above mentioned interactions will lead to weakening of the solid bridges that make the expanded structure of the extrudate, resulting in extruded product that will collapse easily on hydration (or in-mouth due to saliva).

### Analysis

### SDS-PAGE

Extrudate powders from different samples were analyzed by SDS-PAGE both under non-reducing and reducing conditions following the procedure of Laemmli (1970). The samples (1% w/v) were dissolved in buffer containing 62.5 mM Tris-HCl, 25% glycerol, 2% SDS and 0.01% bromophenol blue, pH 6.8. Buffer containing 5% β-mercaptoethanol (β-ME) was used in reducing conditions. The gel was run on a Mini-PROTEAN Tetra Cell (Bio-Rad Laboratories Inc., Hercules, CA, USA). Samples (5 μL in each lane) were run on 4% stacking gel and a 20% separating gel (Mini-PROTEAN Precast gel). The running buffer contained 20% Tris and 5% glycine, pH 6.8. The gel was run for 35 min at a constant voltage of 200 V. Staining was carried out using Coomassie brilliant blue Stain R-250 for 1 h and then the gel was de-stained with de-staining solution (methanol, glacial acetic acid and DI water mixed in ratio of 40:10:50). Precision Plus Protein™ Standards from Bio-Rad Laboratories containing recombinant proteins in the molecular weight range of 10 to 250 kDa were used as protein markers.

### Rehydration Characteristics

### Percent Water Uptake

Extrudates were stored in an evacuated desiccator for 72 h over saturated solutions of lithium chloride (aw: 0.11). The water activity equilibrated extrudate samples of known mass (approx. 2 g) were immersed in 50 g of deionized water at 37° C. The rehydrated samples were taken out of water and weighed at 5, 10, 20, 30 and 40 s. The % water absorption was expressed as weight gain during hydration period per gram extrudate. The data was plotted with time. All analyzes were done in triplicate.

Compression Strength with Hydration

Compression strength of unhydrated and hydrated MPC extrudates was measured using a TA-XT2 texture analyzer operating with Texture Exponent 32 software (Micro Systems, Godalming, United Kingdom) using a 35-mm Perspex cylinder at 50% strain at a test speed of 0.5 mm/s. Extrudate samples were immersed in 50 g of deionized water at 37° C. and compression strength was measured every 10 s from 0 to 50 sec of hydration period. Each measurement was performed with 10 replicates.

### Protein-Protein Interactions

Protein-protein interactions in extruded MPC were determined by analyzing the extractable protein by using selective agents that had the ability to disrupt non-covalent and covalent (disulfide) protein interactions (Liu & Hseih, 2008). Samples were extracted with 0.2M sodium phosphate buffer (PB) solution (pH 6.9) to disrupt electrostatic interactions. Non-covalent interactions were disrupted using PB with 17.3 mM sodium dodecyl sulfate (SDS) and 8M urea. Covalent interactions were reduced in addition to non-covalent interactions using PB with 17.3 mM SDS, 8M urea, and 10 mM of the reducing agent dithiothreitol (DTT) as selective agents. 10 ml of each solvent solution was added to 100 mg ground extruded sample. After vortexing, the buffered samples were kept for 1 h on a rotary shaker at 200 rpm. Samples were centrifuged at 4637×g for 50 min after the extraction step and the supernatants were taken for analysis of the extractable protein content. The absorption of the supernatants was measured at 280 nm using a UV/Vis spectrophotometer (Thermo Fisher Scientific Inc., Waltham, USA). The extractable protein content was calculated from a calibration curve using bovine serum albumin (BSA) as protein standard. Results were expressed as the percentage of extractable protein content of the total protein content present in the sample. The analysis was done in triplicates yielding total nine values per extruded sample.

### Free Sulfhydryl Content

The free sulfhydryl groups in protein solutions (1.0%, w/w) will be determined using Ellman's reagent, DTNB (5,5′-dithio-bis-(2-nitrobenzoic acid) according to the methods of Sava et al. (2005).

### Results

### The Role of Sodium Hexametaphosphate and SC—CO₂ in Dissociation of Casein Micelles and Prevention of Casein-Whey Protein Aggregation, Respectively.

Our experiments have shown that extrusion induces protein-protein interactions and result in the formation of high molecular weight protein aggregates. Design of MPC based, quick disintegrating puffs will require minimization of the calcium bridging, non-covalent interactions in casein, and disulfide interactions between β-lg & casein during extrusion. These interactions lead to formation of insoluble high molecular weight aggregates and strengthen the solid bridges in extrudates. Thus, we employ the chelating ability of SHMP and pH reducting capability of SC—CO₂ to prevent protein interactions during extrusion processing.

### Approach & Methods

MPC-S extrudates were prepared with different levels of SHMP (0.4, 0.7 & 1% on feed basis). The effect of addition of SHMP on calcium chelation was studied by FTIR-ATR spectroscopy. The effect of SC—CO2 on the protein-protein interaction was studied by estimating the extractable protein content in different buffers that sequentially cleave ionic, non-covalent and disulfide interactions of extrudates prepared at different levels of CO2 incorporation (0, 0.3, 0.5, 0.7 & 0.9 kg/h). Free sulfhydryl group content of the extrudates was also analyzed.

### Findings

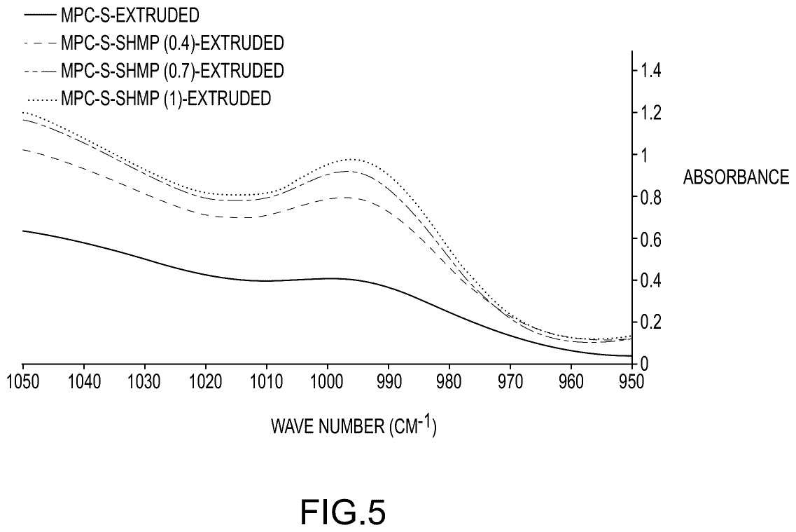

The FTIR spectra show that band intensity at 993 cm-1 that has been associated with ligand free phosphoserine (Gebhardt et al., 2011) increased with increasing SHMP concentration (FIG. 5).

Gebhardt et al. (2011) reported that the bands around 995 and 987 cm-1 suggested changes in stretching vibrations of phosphate moiety of the serine-phosphate residue. The appearance of the two bands were related to release of colloidal calcium phosphate (CCP) from the phosphate residues and its dissociation into Ca2+ and HPO42−, resulting in an increased negative charge of the casein molecules. They concluded that the increments in these intensities occur in parallel to the dissociation of the micelles.

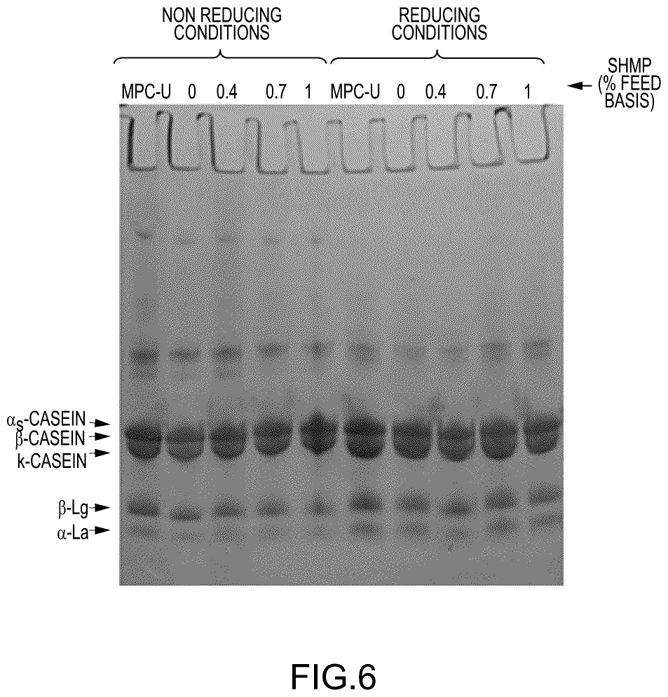

The electrophoretic pattern (FIG. 6) show that the band intensity of casein slightly increased with addition of SHMP (lane 3, 4 & 5) as compared to lane 2 (MPC-S without added SHMP). This indicates that SHMP incorporation in MPC feeds during SCFX prevents protein aggregation possibly by the mechanism as explained by McCarthy et al (2017) and Power et al. (2020).

### Water Absorption Rate

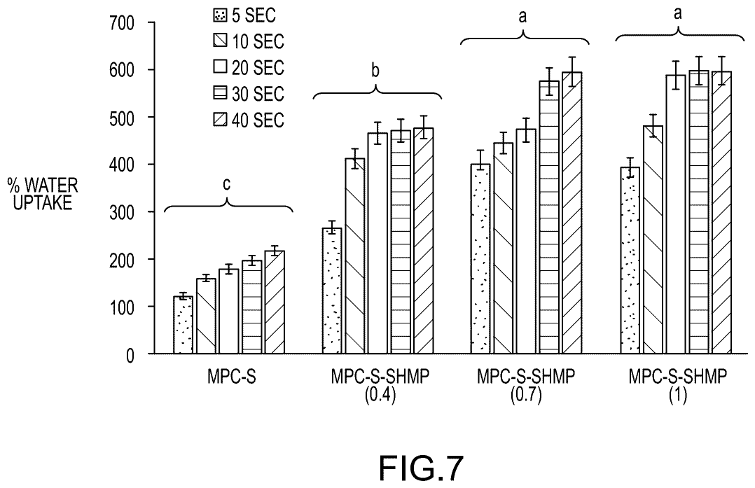

Zuleger and Lippold (2001) stated that hydration kinetics is a major factor affecting tablet disintegration. Thus, rapid rehydration is a crucial property for quick disintegrating puffs. Increase in level of SHMP increased the water absorption of extrudates i.e. demonstrated a higher rate of water uptake (FIG. 7). This may be attributed to increased water affinity of proteins due to SHMP. As the main cause for poor rehydration of MPC powders is casein micelle fusion (Anema et al. 2006; Havea 2006), dissociation of casein micelles due to calcium chelation by SHMP may have improved the water absorption properties of proteins. Rasouli et al. (2019) also reported that high hygroscopicity of SHMP enables it to easily entrap water molecules and thus contribute to the stability of whey proteins in beverages. Power et al. (2020) studied the effect of SHMP on physicochemical properties of transglutaminase crosslinked casein and reported that even in absence of casein dissociation, MPC powders containing SHMP were observed to have increased water sorption compared to MPC powders without SHMP addition. Cunha & Viotto (2010) stated that polyphosphates chelate calcium that creates instability and results in breakdown of casein micelles into smaller soluble submicelles with high water affinity.

Compression Strength with Hydration

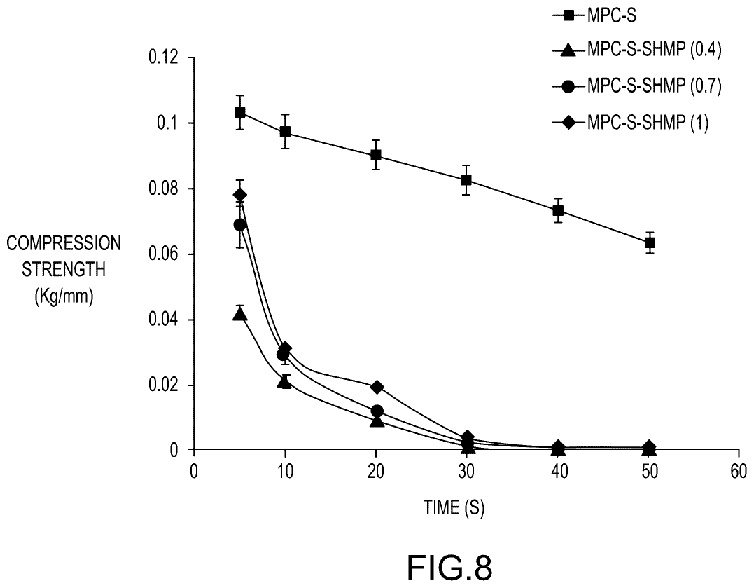

Energy is required for structure breakdown of the extrudate that depends on the state of solid bridges within the porous material which influences the compression strength of the material (Forny et al., 2011). Hydration reduces the strength of solid bridges. For a product to be quick disintegrating, the steep decline in strength of solid bridges is desirable. FIG. 8 demonstrates the compression strength of samples with hydration time. Compression strength of all the samples decreased significantly with increase in hydration time. This decrease can be attributed to water absorption by the extrudates resulting in softening of the matrix. Higher compression strength was observed for extrudates without SHMP throughout the hydration time as compared to extrudates prepared with SHMP in the formulation. Increase in SHMP concentration in formulations led to a corresponding decrease in compression strength. This can be attributed to increased water absorption by extrudates containing higher levels of SHMP, resulting in weakening of the extrudate structure and decreased mechanical strength. These results are coherent with rehydration analysis and conclude that faster rehydration leads to faster lowering of compression strength and thus easier disintegration.

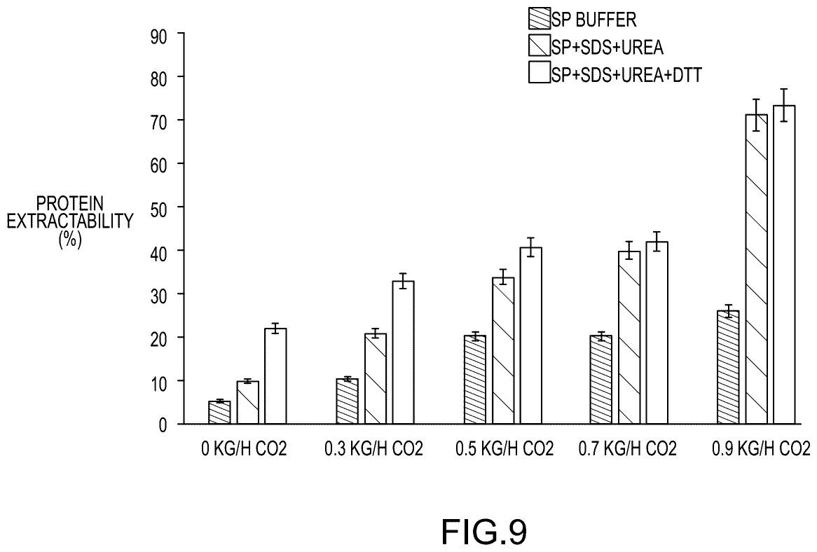

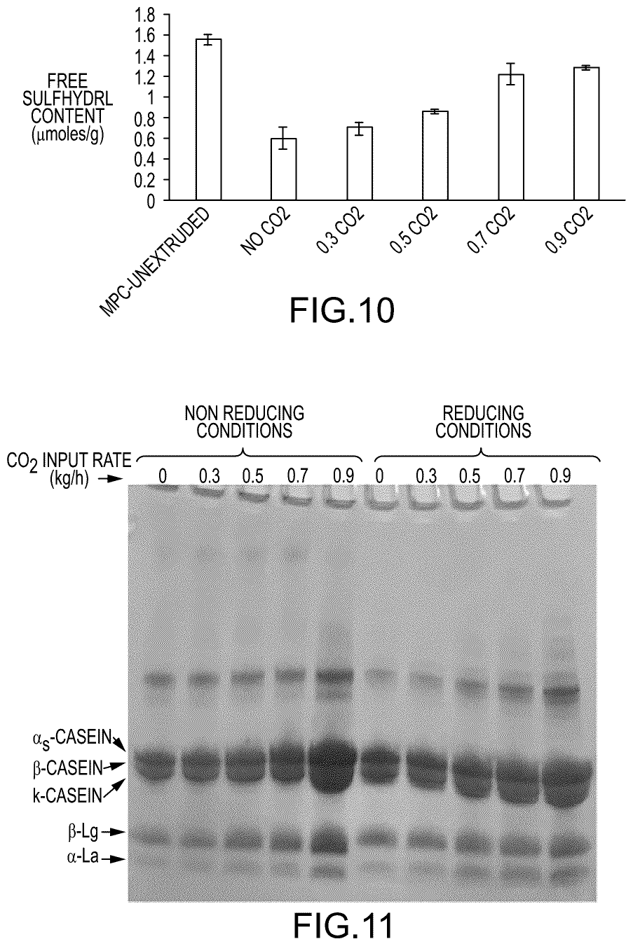

Effect of SC—CO2 incorporation on the protein-protein interaction (FIG. 9) and free sulfhydryl content (FIG. 10) of extrudates indicate that SC—CO₂ prevents the sulfhydryl interactions. Protein-protein interaction study show that no significant sulfhydryl interactions occur at SC—CO2>0.7 kg/h. Similarly, free sulfhydryl content study also demonstrate that with increase in CO2 incorporation rate free sulfhydryl content increases. As β-globulin and k-casein aggregation is dependent on the formation of disulfide bonds, this indicates that SC—CO2 incorporation during extrusion can prevent casein-whey protein aggregation.

Electrophoretic patterns (FIG. 11) demonstrate that with increase in SC—CO2 input rate during SCFX, interactions between casein and whey proteins decrease as evident by increase in band thickness indicating increased solubility of casein proteins (as, β) and whey proteins (β-Lg & α-La) from lane 1-5 (0 to 0.9 kg/h SC—CO2 input rate). Similar trend is observed in reducing conditions.

Thus, incorporation of SC—CO2 (through SCFX) and SHMP in the extruder feed can prevent the sulfhydryl interactions and calcium bridging, respectively during extrusion. This was utilized to prepare milk protein based extrudates with quick disintegrating characteristics.

### Example 2

### Supercritical Fluid Extrusion: Elucidating the Physical and Reactive Role of Supercritical Carbon Dioxide During Extrusion of Milk Protein Concentrate

### Introduction

In steam extrusion (SX), a heterophase, low-moisture (<22 wt. %) polymer melt is brought to high temperatures (>130° C.) and pressures which upon exiting through a die, is puffed by rapid conversion of compressed water to steam. Contrastingly, in supercritical fluid extrusion (SCFX), the dual role of water, as a plasticizer and blowing agent in conventional extrusion, is decoupled since the expanded structure formation is through monitored pressure drop resulting in CO₂ based nucleation and subsequent diffusion-controlled expansion, and hence expansion can be achieved at lower temperature (<100° C.). Moreover, due to the pressure-dependent solubility of SC—CO₂ in melts, SCFX can also be used as a reactor to adjust the pH by formation of carbonic acid and modify the in-barrel reactions.

### Aim

The effects of SX and SCFX (with variable SC—CO₂ injection rates) on the structural and physicochemical properties of extruded MPC85 were investigated to obtain fundamental information on the reactive role of SC—CO₂ during SCFX.

### Methods

SX and SCFX experiments were performed using MPC85 feed. The OLI stream analyzer was adopted to determine the pH and pressure dependence of aqueous CO₂ concentration. Protein interactions during extrusion were evaluated by analyzing the protein extractability, SDS-PAGE, and free sulfhydryl content. Particle size, zeta (ζ) potential, and viscosity of powdered extrudates were analyzed to evaluate the physicochemical properties of extrudates.

### Results

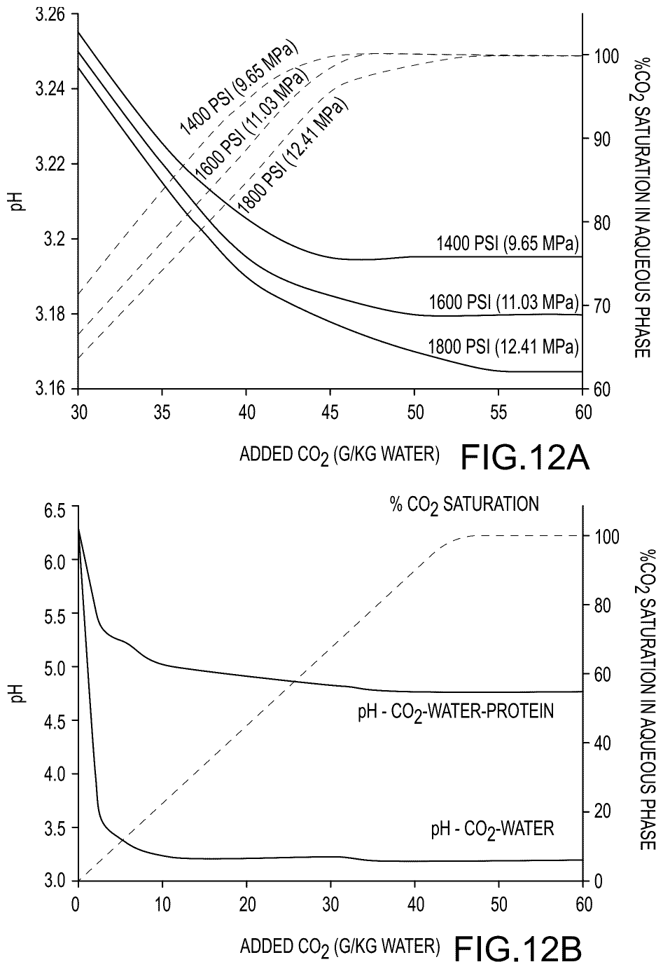

Thermodynamic simulation demonstrated that the saturation CO₂ concentration in the melt increased with increasing pressure and correspondingly decreased the pH of the aqueous phase (FIG. 12A). The decreased effect of CO₂ concentration on the simulated pH (4.85) of the CO₂—H₂O-protein system compared to the pH (3.25) of the CO₂—H₂O system can be attributed to the buffering capacity of dairy proteins (FIG. 12B).

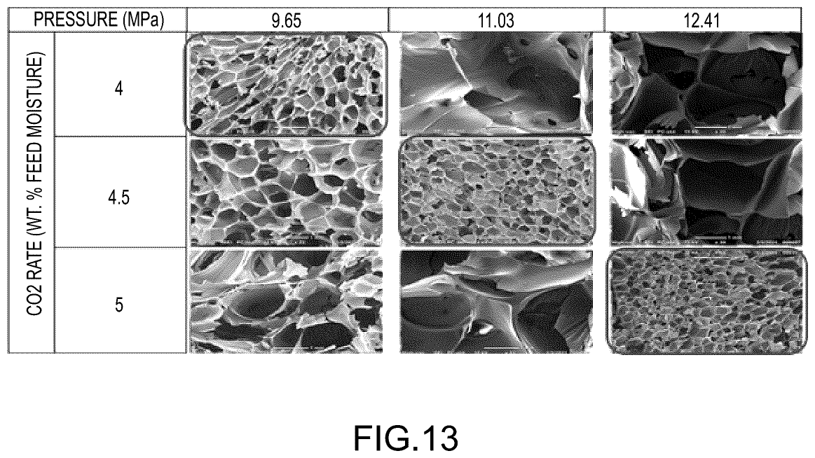

The SEM images (FIG. 13) show that the extrudates generated using SC—CO₂ corresponding to the saturation level in dough moisture at operating pressure exhibited a uniform cellular morphology, whereas large cells and a non uniform cellular structure was observed for samples prepared with undersaturated or oversaturated SC—CO₂ conditions.

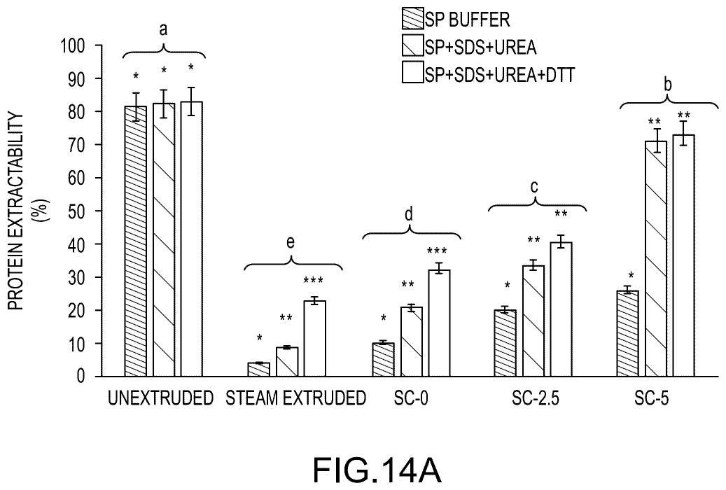

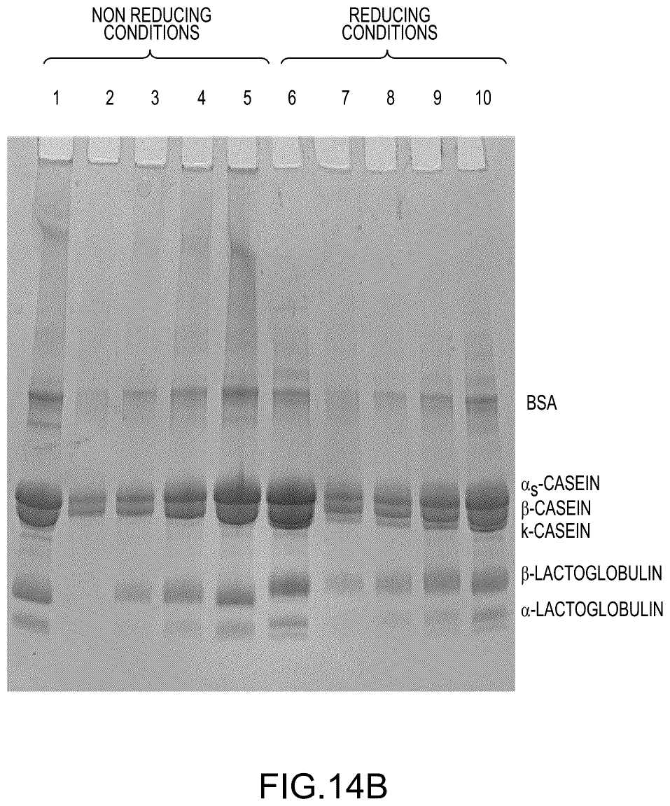

Increasing the SC—CO₂ input rate from 0 to 5 wt. % feed moisture resulted in enhanced protein extractability in the buffers (FIG. 14A). Similar results were validated by electrophoretic patterns (FIG. 14B).

The protein extractability increased by 536.8, 621.7 and 223.3% for SP, SP+SDS+Urea and SP+SDS+Urea+DTT, respectively and free sulfhydryl content increased from 0.59 to 1.28 μmoles/g protein when SC—CO₂ input rate was increased from 0 to 5 (wt. % of feed moisture).

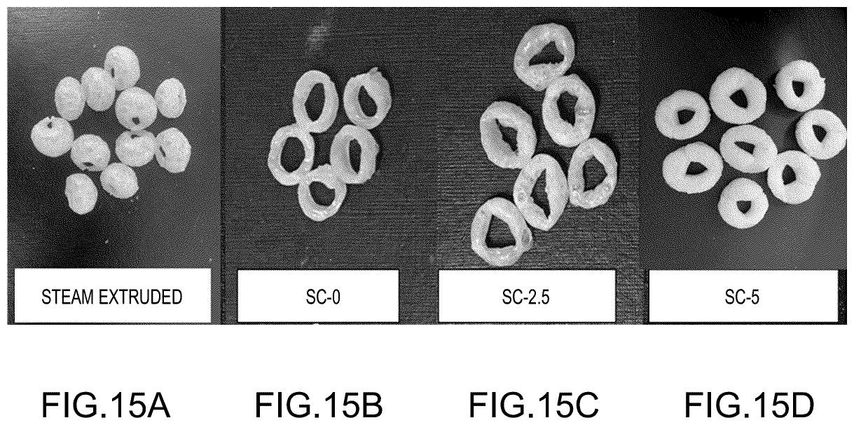

Table 1 describes the extrusion operational parameters and selected product characteristics for steam extrusion (SX) and supercritical fluid extrusion (SCFX) of milk protein concentrate. FIG. 15 shows MPC extrudates prepared by SX and SCFX.

TABLE 1

| | | | | Mean | |
|---|---|---|---|---|---|
| | | | | particle | Zeta |
| | Extrusion parameters | | | size - | potential - |
| | | SC-CO₂ | Product | soluble | soluble |
| | Extrusion | (wt. % feed | Temperature | protein | protein |
| Sample | Type | moisture) | (° C.) | (nm) | (mV) |
| Unextruded | — | — | — | 232 ± 2.08^{bc} | −24.1 ± 3.72^{ab} |
| SX | Steam | — | 115 | 566 ± 4.8^{e} | −12.8 ± 0.99^{d} |
| SC-0 | SCFX | 0 | 85 | 312 ± 6.5^{d} | −13.6 ± 1.43^{d} |
| SC-2.5 | SFCX | 2.5 | 85 | 218 ± 2.6^{b} | −22.2 ± 2.27^{c} |
| SC-5 | SCFX | 5 | 85 | 166 ± 1.6^{a} | −28 ± 1.23^{a} |

### Conclusions

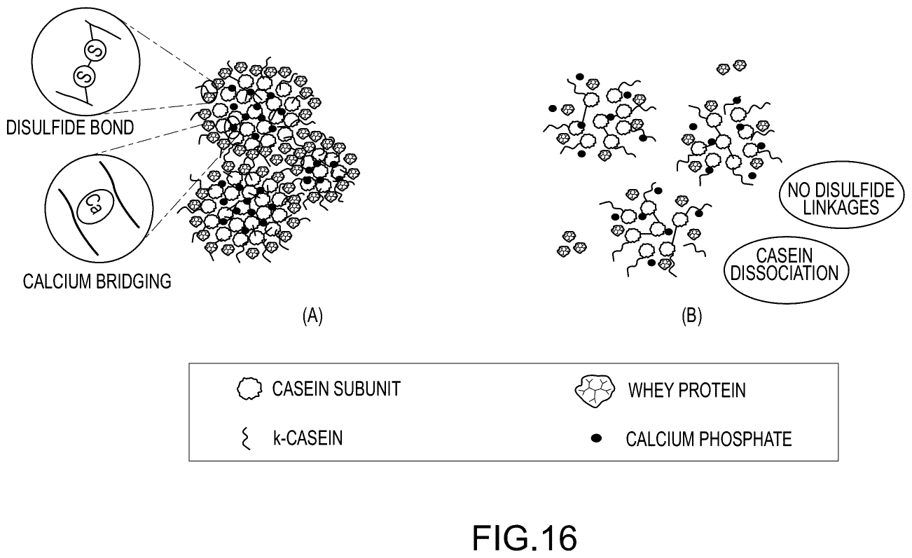

SC—CO₂ input rate corresponding to the extruder operating pressure to ensure a saturated system (dissolved CO₂ in aqueous part of formulation) in the barrel is required for morphologically uniform extrudates of a priori designed quality. FIG. 16 is a schematic showing milk protein aggregation during steam (A) and SCF extrusion (B).

The study also established that lower temperature and temporarily induced acidity due to the formation of carbonic acid during SCFX decreased protein-protein interactions in extruded milk proteins and contributed to the improved physicochemical properties of the extrudates.

### Example 3

### In-Mouth Self-Disintegrating Milk Protein Puffs Prepared by Reactive Supercritical Fluid Extrusion

### Introduction

In-mouth disintegrating puffed cereals have recently become popular finger foods for toddlers that offer the convenience of self-feeding, portability, and a mess-free eating environment. However, there is an unmet demand for protein-rich products with similar attributes. In this study, temporarily induced acidity due to the formation of carbonic acid during low-temperature, low-shear supercritical fluid extrusion (SCFX) along with incorporation of sodium hexametaphosphate (SHMP) as chelating agent was employed to prepare cheerio-type milk protein concentrate (MPC80) based puffed extrudates that disintegrated in-mouth in less than 30 s.

### Aim

To study the effects of steam extrusion (SX) and SCFX (with and without addition of SHMP) on the physicochemical, hydration and disintegration properties of MPC based extruded puffs.

### Methods

Steam extrusion (SX) and SCFX were used to prepare MPC-80-sucrose (MPC-80-S) extrudates with and without added SHMP. SDS-PAGE was done to evaluate the protein interactions that occurred during processing. Compression strength with hydration, and artificial tongue simulation (two bite test) of extrudates was done to objectively evaluate their disintegration behavior.

### Results

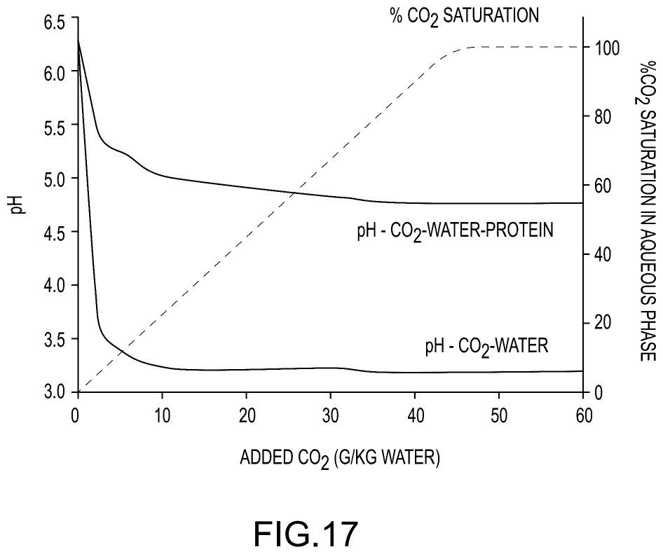

The pH of the CO₂—H₂O-protein system was simulated to be 4.8 compared to 3.2 for the CO₂—H₂O system. The decreased effect of CO₂ concentration on the simulated pH of the CO₂—H₂O-protein system is predicated to be contributed by the buffering capacity of dairy proteins (FIG. 17).

Calcium chelation by SHMP and temporarily induced acidity (pH 4.85) due to SC—CO₂ incorporation during SCFX minimized the protein-protein interactions due to calcium bridging and disulfide bonding, respectively.

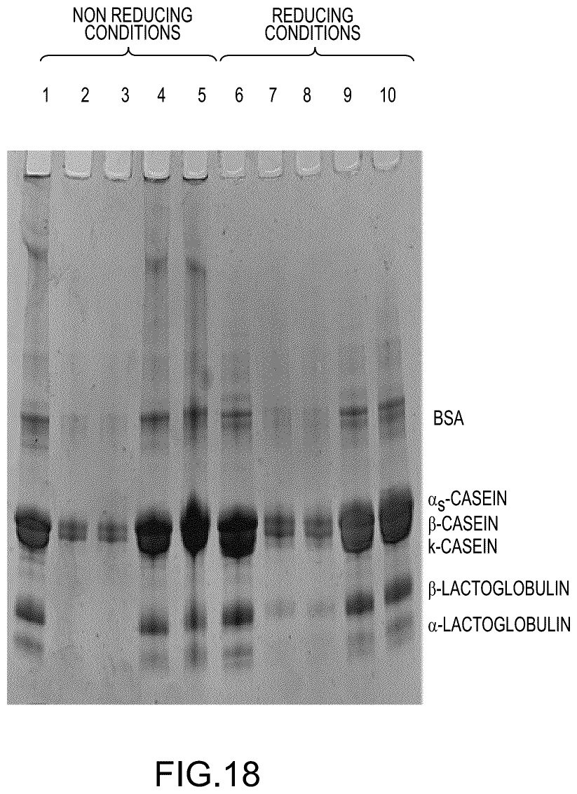

The electrophoretic patterns demonstrated increased solubility of caseins in SCFX-SHMP extrudates when compared to SCFX-control (FIG. 18). SX samples did not demonstrate any increase in solubility in SDS PAGE analysis.

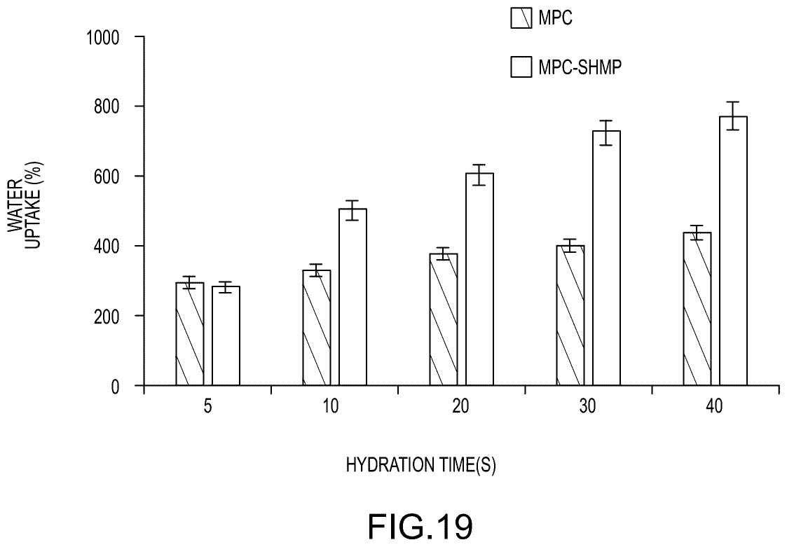

SCFX-SHMP extrudates absorbed significantly more water than SCFX-control extrudates after 30 s of hydration (FIG. 19).

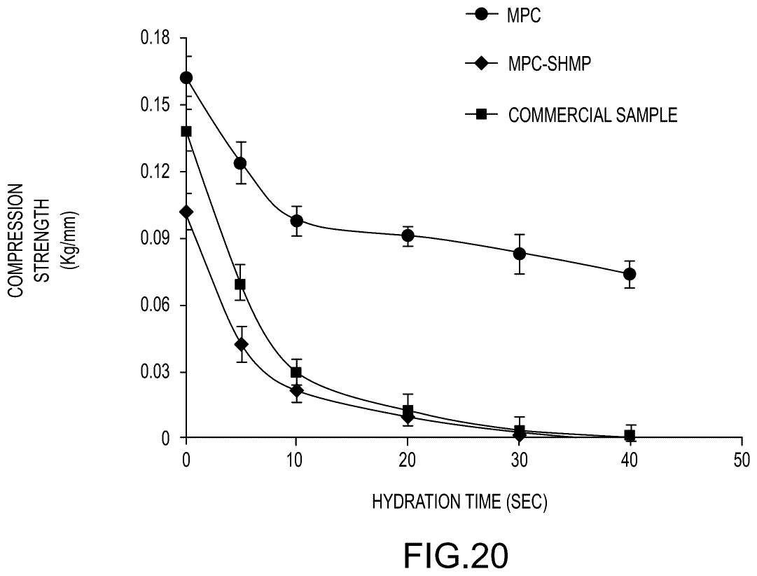

Compression strength of SCFX-SHMP extrudates reduced by 93% on hydration (similar to commercial starch-based samples) as compared to 31% for SCFX-control samples (FIG. 20).

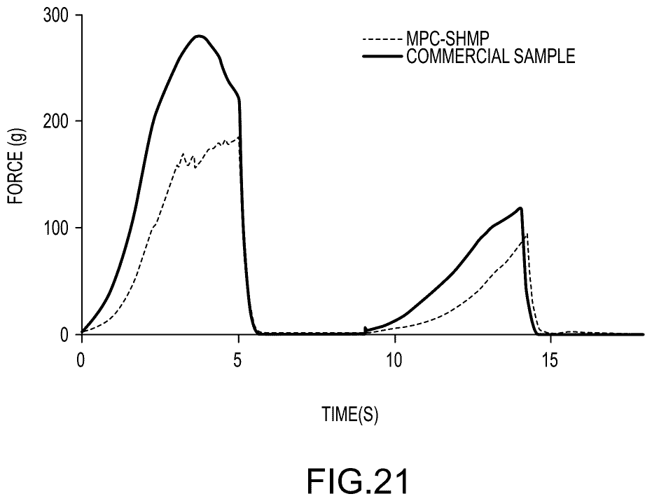

During hydration, increased water absorption by SCFX-SHMP extrudates resulted in weakened extrudate structure and decreased mechanical strength contributing to easy disintegration quantitatively analyzed by two bite test (FIG. 21), and qualitatively observed to be less than 30 s in the mouth.

High temperature and absence of SC—CO₂ in SX-SHMP and SX-control samples resulted in significant protein aggregation and the prepared extrudates lacked self-disintegration behavior.

Table 2 shows proximate composition (7 g serving) of MPC-based puffs and commercially available starch-based product.

TABLE 2

| | MPC | Commercial |
|---|---|---|
| Amount per serving | product | product |
| Calories (kcal) | 25 | 25 |
| Protein (g) | 4.6 | 0 |
| Total Fat (g) | 0.08 | 0 |
| Total Carbohydrates | 1.24 (1) | 6 (1) |
| (Added sugar)-g | | |

The values are calculated (not experimental)

### Conclusions

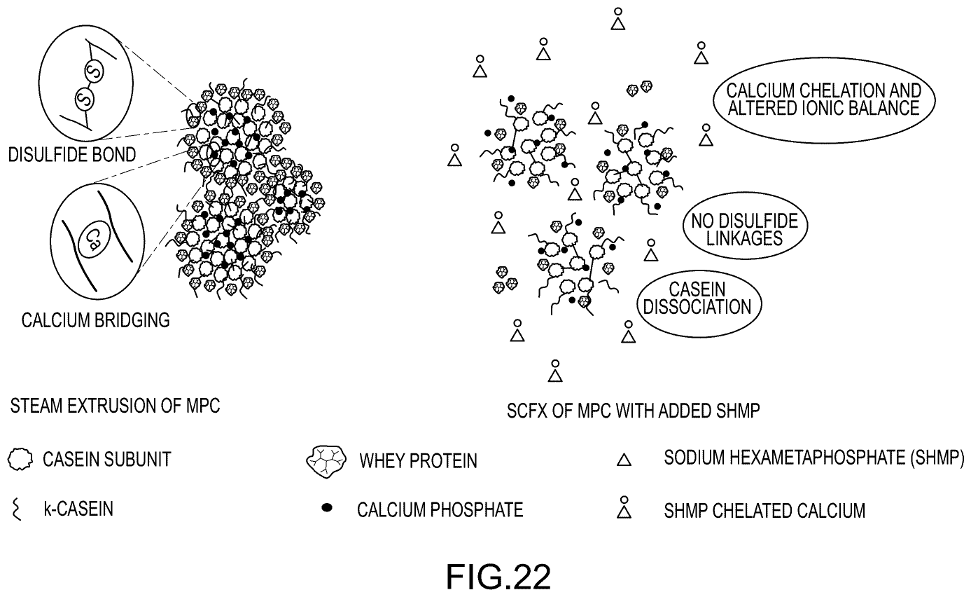

This study elucidated the reactive role of SC—CO₂ in SCFX along with calcium chelation by SHMP in altering the physicochemical properties of MPC-80 to help generate expanded extrudates of defined microstructure that self-disintegrate in the mouth in less than 30 s. FIG. 22 is a schematic of protein aggregation and the effect of incorporation of SHMP in MPC extrudates prepared by SCFX vs SX.

The texture modified extrudates generated by the reported breakthrough process offers the following advantages:
(a) Promising protein-rich alternative to starch-based finger foods used for baby led weaning of toddlers
(b) Easy to carry and does not require water addition
(c) Can be designed with desired micronutrient profiles for targeted populations
(d) Suitable for elderly and dysphagic patients or patients with other conditions that make chewing and swallowing difficult.

### Example 4

### Consumer Evaluation

### Consumer Acceptance Study Protocol

The consumer acceptability attributes of the prepared in-mouth disintegrating milk protein baby puff were compared with commercial starch based baby puffs by sensory evaluation. An untrained sensory panel was selected from an existing pool of panelists having varying levels of familiarity with the puffed baby food and with no dairy/lactose allergies. Panelists were recruited at random (N=42) and demographic data (age, gender, familiarity with the product) were collected. The panelists were provided with study instructions and samples each of commercial starch-based baby puffs, experimental milk protein baby puffs and commercial milk protein puffs. Spring water was also provided as palate cleanser to be used between tasting of samples. The panelists were asked to electronically record their consent and responses (RedJade software, Curion Insights, Redwood City, CA, USA) on the time taken to disintegrate the sample, time dependent disintegration profile, texture, sweetness and other attributes pertaining to the baby puff characteristics using a disintegration timer, a rider chart and a 5-point just about right (JAR) scale. A start-stop timer test was used to record in-mouth disintegration time for the puffed product. A 5-point JAR scale has ‘too much’ or ‘too little’ as opposite end anchors, with the center point being ‘just about right’. These scales were used to quantify the panelists perception on a specific attribute. Sensory experiments were conducted according to the protocols approved by the Institutional Review Board for Human Participants at Cornell University, US.

### Consumer Acceptance Study Results

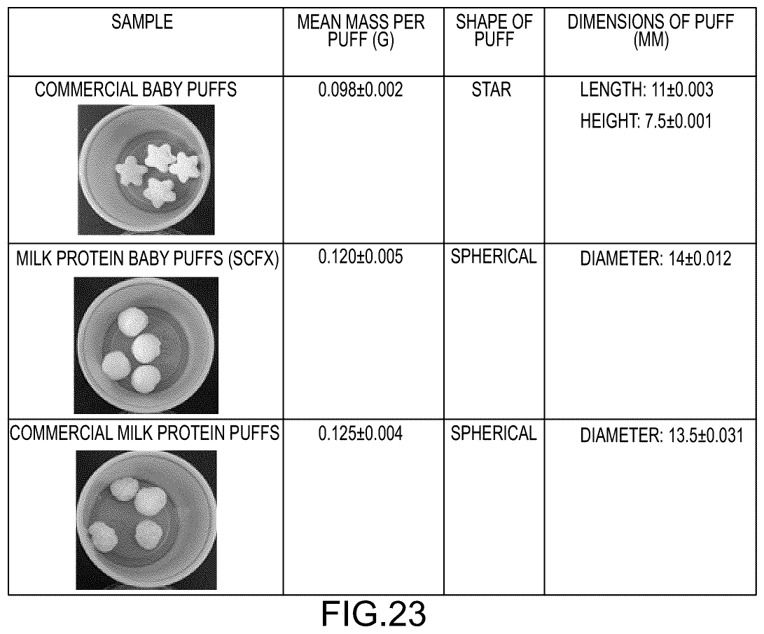

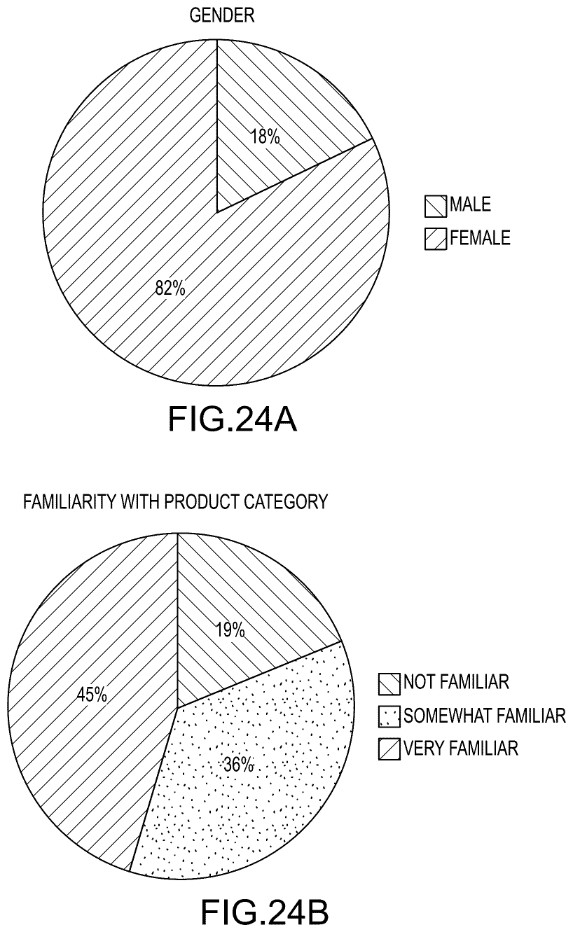

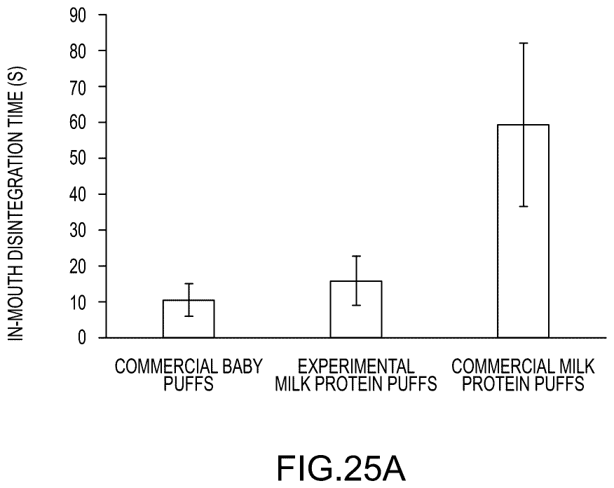

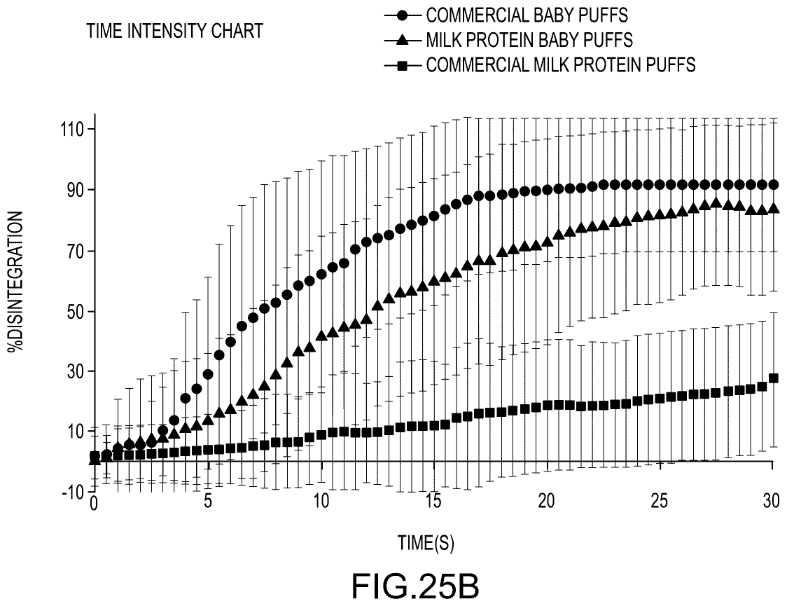

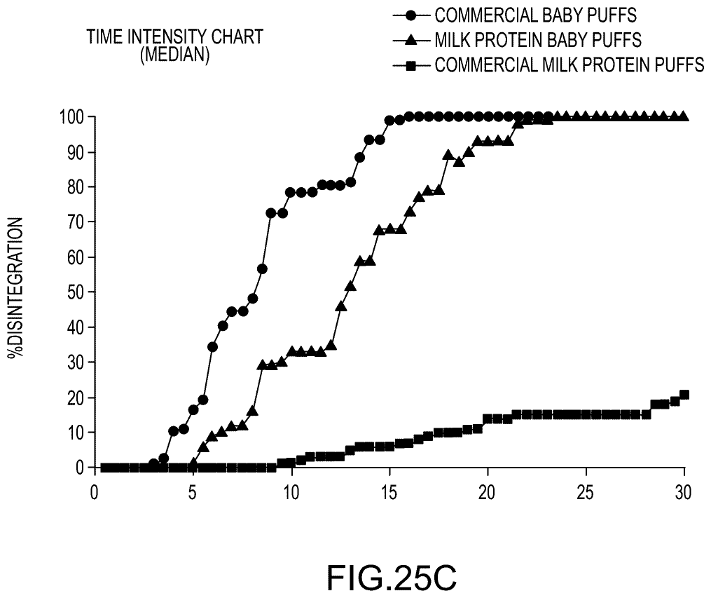

The samples served to the panelists along with their dimensional data is demonstrated in FIG. 23. The demographic data of the recruited panelists (gender, familiarity with the product category) are shown in FIG. 24. Responses showed that 82% of the panelists were female and out of all panelists 81% of them were familiar with the product category. Mean in-mouth disintegration time of the samples is shown in FIG. 25A. The commercial starch-based baby puff demonstrated fastest in-mouth disintegration time of 11 s followed by the milk protein-based baby puff that disintegrated in 16 s. The commercial milk protein puff took >58 s to disintegrate, even though they were very similar in composition to MPC puffs prepared by SCFX. Although the dimensions of MPC based baby puffs were larger (more penetration depth) than commercial baby puffs but, they were highly comparable to the commercial baby puffs in disintegration characteristics and dissolved in less than 30 s in the mouth. This signifies the utility of supercritical fluid extrusion along with SHMP incorporation in MPC feeds for the preparation of in-mouth disintegrating baby puffs as compared to conventional steam extrusion. The time dependent disintegration profile of the extrudate samples is shown in FIG. 25B (mean) and FIG. 25C (median). The disintegration profile of commercial baby puffs and milk protein baby puffs manufactured by SCFX was comparable with both the products achieving >80% disintegration in 30 s (mean data). In contrast the commercial milk protein puff demonstrated 33% disintegration in 30 s of evaluation.

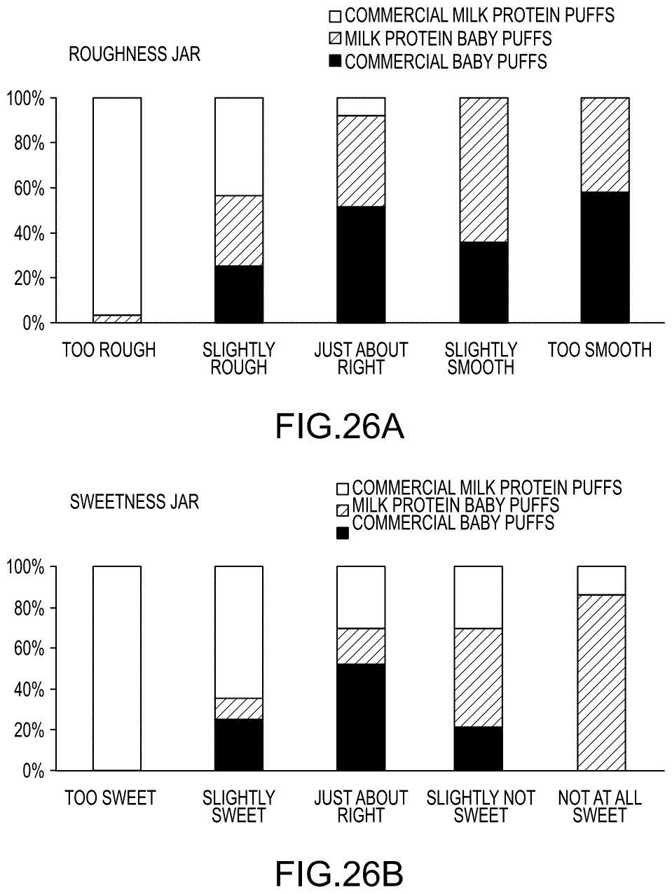

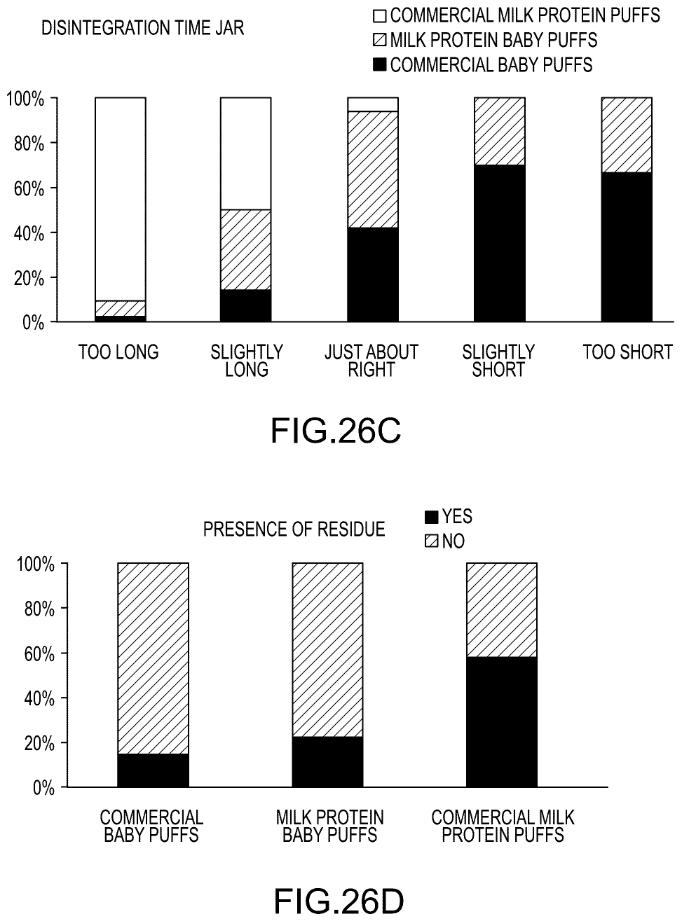

FIGS. 26A-26D demonstrate the JAR results for the product attribute, namely, roughness/smoothness (FIG. 26A), sweetness (FIG. 26B), disintegration time (FIG. 26C) and, the presence or absence of residue after swallowing (FIG. 26D). On the roughness attribute, more than 50% of the panelists reported who ‘just about right’ reported that for commercial baby puffs followed by 40% for milk protein baby puffs. Almost all the respondents (98%) who reported the product to be ‘too rough’ to be used as a baby food reported it for commercial milk protein puffs. Milk protein baby puff developed by SCFX was reported in less sweet category in comparison to the commercial baby puff and commercial milk protein puff. More than 52% of the panelists reported just about right for disintegration time of MPC based baby puffs followed by 42% for commercial baby puffs. The commercial milk protein puffs were reported to take either slightly or too long disintegration time by 98% of the panelists. Nearly 16 and 22% of the panelists reported the presence of residue after swallowing for commercial baby puffs and milk protein baby puffs, respectively. In contrast, 60% of the panelists reported the presence of residue after swallowing in the commercially available milk protein puffs. These results establish the advantage of SCFX and incorporation of SHMP in MPC feed for the generation of in-mouth disintegrating milk protein based puffed food suitable for consumption by toddlers.

### REFERENCES

Citation of a reference herein shall not be construed as an admission that such reference is prior art to the present invention. All references cited herein are hereby incorporated by reference in their entirety. Below is a listing of various references cited herein:
Arora, B., Schulz, P., & Rizvi, S. S. H. (2020). In-process flow behavior and expansion characteristics of milk protein extrudates. Journal of Food Processing and Preservation, e15348.
Brown, A., & Lee, M. D. (2015). Early influences on child satiety-responsiveness: the role of weaning style. Pediatric Obesity, 10, 57-66.
Cadesky, L., Walkling-Ribeiro, M., Kriner, K. T., Karwe, M. V., & Moraru, C. I. (2017). Structural changes induced by high-pressure processing in micellar casein and milk protein concentrates. Journal of Dairy Science, 100(9), 7055-7070.
Cameron, S. L., Heath, A. L., Taylor, R. W. (2012). How feasible is baby-led weaning as an approach to infant feeding? A review of the evidence. Nutrients, 4, 1575-1609.
Carr A., Golding M. (2016) Functional Milk Proteins Production and Utilization: Casein-Based Ingredients. In: McSweeney P., O'Mahony J. (eds) Advanced Dairy Chemistry. Springer, New York, NY
Cho, K. Y., & Rizvi, S. S. H. (2009a). 3D microstructure of supercritical fluid extrudates I: Melt rheology and microstructure formation. Food Res. Int'l 42, 595-602.
Cho, K. Y., & Rizvi, S. S. H. (2009b). 3D Microstructure of supercritical fluid extrudates. II: Cell anisotropy and the mechanical properties. Food Res. Int'l, 42, 603-611.
Havea, P. (2006). Protein interactions in milk protein concentrate powders. International Dairy Journal, 16(5), 415-422.
Loret, C., Walter, M., Pineau, N., Peyron, M. A., Hartmann, C., & Martin, N. (2011). Physical and related sensory properties of a swallowable bolus. Physiol & Behav, 104, 855-864.
McCarthy, N. A., Power, O., Wijayanti, H. B., Kelly, P. M., Mao, L., & Fenelon, M. A. (2017). Effects of calcium chelating agents on the solubility of milk protein concentrate. International Journal of Dairy Technology, 70(3), 415-423.
Molins, R. A. (1991). Phosphates in Food. CRC Press, Boca Raton, FL.
Motoi, L., Morgenstern, M. P., Duncan, I., Wilson, A. J. & Balita, S. (2013). Bolus moisture content of solid foods during mastication. Journal of Texture Studies, 44, 468-479.
Rapley, G. (2011). Baby-led weaning: transitioning to solid foods at the baby's own pace. Community Practice, 84(6), 20-23.
Rizvi, S. S. H. & Mulvaney, S. J. (1992). Extrusion Processing with Supercritical Fluids. U.S. Pat. No. 5,120,559, Jun. 9, 1992.
Sikand, V., Tong, P. S., Roy, S., Rodriguez-Saona, L. E., & Murray, B. A. (2011). Solubility of commercial milk protein concentrates and milk protein isolates. Journal of Dairy Science, 94(12), 6194-6202.
WHO (2019). WHO/Europe studies find baby foods are high in sugar and inappropriately marketed for babies. www.euro.who.int/en/media-centre/sections/press releases/2019/whoeurope-studies-find-baby-foods-are-high-in-sugar-and-inappropriately marketed-for-babies

Illustrative embodiments of the processes, methods, and products of the present disclosure are described herein. It should be understood, however, that the description herein of the specific embodiments is not intended to limit the present disclosure to the particular forms disclosed but, on the contrary, the intention is to cover all modifications, equivalents, and alternatives falling within the spirit and scope of the invention by the appended claims. Thus, although the present invention has been described for the purpose of illustration, it is understood that such detail is solely for that purpose and variations can be made by those skilled in the art without departing from the spirit and scope of the invention which is defined by the following claims.

What is claimed is:

1. A method of preparing an orally dissolving protein extrudate product, the method comprising:
(a) blending a protein concentrate with one or more extrusion component to produce a protein dough;
(b) extruding the protein dough with supercritical fluid to form a wet protein extrudate, said extruding being effective to inhibit protein-protein interactions in the protein concentrate; and
(c) drying the wet protein extrudate to form a protein extrudate product that is orally dissolving.

2. The method of claim 1, wherein the protein concentrate comprises milk protein concentrate.

3. The method of claim 2, wherein the one or more extrusion component comprises one or more sugar, emulsifier, and/or dispersing agent.

4. The method of claim 3, wherein the sugar comprises one or more saccharide selected from the group consisting of monosaccharides, disaccharides, and oligosaccharides, wherein the monosaccharides comprise glucose and/or fructose, the disaccharides comprise sucrose and/or maltose, and the oligosaccharides comprise maltodextrins.

5. The method of claim 4, wherein the saccharide is sucrose.

6. The method of claim 5, wherein the emulsifier is selected from the group consisting of lecithin, distilled monoglycerides, polysorbates, and diacetyl tartaric esters of mono and diglycerides.

7. The method of claim 6, wherein the dispersing agent is a calcium chelating agent selected from the group consisting of sodium hexametaphosphate (SHMP), trisodium diphosphate, and tetrasodium diphosphate.

8. The method of claim 7, wherein the dispersing agent is sodium hexametaphosphate (SHMP).

9. The method of claim 1, wherein the protein dough comprises:
(a) protein concentrate at about 40-80 wt %, or at about 50-70 wt %, or at about 60 wt %;
(b) sugar at about 2-20 wt %, or at about 5-15 wt %, or at about 9.6 wt %;
(c) emulsifier(s) at about 0-3 wt %, or at about 0.5-2 wt %, or at about 1.42 wt %;
(d) dispersing agent at about 0.1-0.5 wt %, or at about 0.3-0.5 wt %, or at about 0.357 wt %; and
(e) water at about 20-40 wt %, or at about 25-35 wt %, or at about 28.5 wt %.

10. The method of claim 1, wherein the protein dough comprises about 840 kg of protein concentrate, about 135 kg of saccharide, about 10 kg of lecithin, about 10 kg of distilled monoglycerides, and about 5 kg of dispersing agents.

11. The method of claim 1, wherein the extruding the protein dough with supercritical fluid comprises a feed rate of about 1-50 kg/h.

12. The method of claim 1, wherein the extruding the protein dough with supercritical fluid comprises a feed rate of about 35 kg/h.

13. The method of claim 1, wherein the extruding the protein dough with supercritical fluid comprises a screw speed of about 1-200 rpm.

14. The method of claim 1, wherein the extruding the protein dough with supercritical fluid comprises a screw speed of about 110 rpm.

15. The method of claim 1, wherein the extruding the protein dough with supercritical fluid comprises a supercritical fluid injection pressure of about 1 to 20 MPa.

16. The method of claim 1, wherein the extruding the protein dough with supercritical fluid comprises a supercritical fluid injection pressure of about 11 MPa.

17. The method of claim 1, wherein the extruding the protein dough with supercritical fluid comprises a barrel set temperature of about 5 to 40° C.

18. The method of claim 1, wherein the extruding the protein dough with supercritical fluid comprises supercritical fluid feed rate of about 0.8 kg/h.

19. The method of claim 1, wherein the extruding the protein dough with supercritical fluid comprises a water feed rate of about 14 kg/h.

20. The method of claim 1, wherein the supercritical fluid is carbon dioxide.

21. The method of claim 1, wherein the blending comprises dry blending.

22. The method of claim 21, wherein the dry blending comprises blending in a ribbon blender for about 5-30 minutes, or for about 10, 15, 20, 25, or 30 minutes.

23. The method of claim 1, wherein the wet protein extrudate is collected at a temperature of between about 70-95° C., or about 70° C., 75° C., 80° C., 85° C., 90° C., or 95° C.

24. The method of claim 1, wherein the wet protein extrudate is collected at a temperature of about 85° C.

25. The method of claim 1, wherein the drying comprises conventional oven drying or vacuum oven drying.

26. The method of claim 1, wherein the drying comprises drying until a moisture content of the protein extrudate product is less than about 5-15%, or about 5%, 10%, or 15%.

27. The method of claim 1, wherein the drying comprises conventional drying at greater than about 50-95° C. with or without vacuum, or greater than about 50° C., 60° C., 70° C., 80° C., 85° C., 90° C., or 95° C. with or without vacuum.

28. The method of claim 1, wherein the protein extrudate product is fast orally dissolving.

29. The method of claim 1, wherein the protein extrudate product is protein-rich.

30. An orally dissolving protein extrudate product prepared by the method of any one of claims 1-29.

31. The protein extrudate product of claim 30 comprising:
(a) protein concentrate at about 60-90 wt %, or at about 65-85 wt %, or at about 84 wt %;
(b) sugar at about 5-25 wt %, or at about 10-20 wt %, or at about 13.5 wt %;
(c) emulsifier(s) at about 0.05-4 wt %, or at about 1-3 wt %, or at about 1-2 wt %; and
(d) dispersing agent at about 0.1-0.5 wt %, or at about 0.3-0.5 wt %, or at about 0.4-0.5 wt %.

32. The protein extrudate product of claim 30, wherein the protein extrudate product comprises about 84 wt % milk protein concentrate, about 13.5 wt % sucrose, about 1 wt % distilled monoglycerides, and about 0.5 wt % sodium hexametaphosphate.

33. A protein extrudate product comprising:
(a) a protein concentrate;
(b) a sugar;
(c) an emulsifier; and
(d) a dispersing agent comprising a calcium chelating agent,
wherein the protein extrudate product is orally dissolving and protein-rich.

34. The protein extrudate product of claim 33 comprising:
(a) protein concentrate at about 60-90 wt %, or at about 65-85 wt %, or at about 84 wt %;
(b) sugar at about 5-25 wt %, or at about 10-20 wt %, or at about 13.5 wt %;
(c) emulsifier(s) at about 0.05-4 wt %, or at about 1-3 wt %, or at about 1-2 wt %; and
(d) dispersing agent at about 0.1-0.5 wt %, or at about 0.3-0.5 wt %, or at about 0.4-0.5 wt %.

35. The protein extrudate product of claim 34, wherein the protein concentrate comprises a milk protein concentrate.

36. The protein extrudate product of claim 35, wherein the sugar comprises one or more saccharide selected from the group consisting of monosaccharides, disaccharides, and oligosaccharides, wherein the monosaccharides comprise glucose and/or fructose, the disaccharides comprise sucrose and/or maltose, and the oligosaccharides comprise maltodextrins.

37. The protein extrudate product of claim 36, wherein the saccharide is sucrose.

38. The protein extrudate product of claim 37, wherein the emulsifier is a food grade emulsifier selected from the group consisting of lecithin, distilled monoglycerides, polysorbates, and diacetyl tartaric esters of mono and diglycerides.

39. The protein extrudate product of claim 38, wherein the dispersing agent is a calcium chelating agent selected from the group consisting of sodium hexametaphosphate (SHMP), trisodium diphosphate, and tetrasodium diphosphate.

40. The protein extrudate product of claim 39, wherein the dispersing agent is sodium hexametaphosphate (SHMP).

41. The protein extrudate product of claim 33, wherein the protein concentrate is prepared by ultrafiltration.

42. The protein extrudate product of claim 33, wherein the protein concentrate is prepared by diafiltration.

43. The protein extrudate product of claim 33, wherein the protein extrudate product comprises a shelf life of more than about 3-12 months, or more than about 3, 6, 9, or 12 months.

44. The protein extrudate product of claim 33, wherein the protein extrudate product further comprises one or more of a micronutrient, a coloring agent, a flavoring agent, and/or a food grade additive.

45. The protein extrudate product of claim 44, wherein the one or more micronutrient is selected from the group consisting of niacinamide, alpha-tocopheryl acetate, pyridoxine hydrochloride, thiamine hydrochloride, electrolytic iron, zinc, and the like.

46. The protein extrudate product of claim 44, wherein the one or more coloring agent is a food grade coloring agent selected from the group consisting of carotenoids, betalains, curcumin, and the like.

47. The protein extrudate product of claim 44, wherein the one or more flavoring agents is a food grade flavoring agent selected from the group consisting of natural flavoring (for example, natural vanilla flavor), artificial flavoring, fruit puree (e.g., dried blueberry puree, dried apple puree, etc.), and the like.

48. The protein extrudate product of claim 44, wherein the one or more food grade additive is selected from the group consisting of mixed tocopherols, lecithin, dimodan, datem, and the like.

49. The protein extrudate product of claim 33, further comprising one or more of:
(a) micronutrient(s) at about 0.00004-0.0004 wt %, or at about 0.00005-0.0035 wt %, or at about 0.0005 wt %;
(b) coloring agents at about 0.5-3 wt %, or at about 1-2 wt %, or at about 1.5 wt %;
(c) flavoring agent(s) at about 0.5-3 wt %, or at about 1-2 wt %, or at about 1.5 wt %; and/or
(d) food grade additive(s) at about 0.5-1.5 wt %, or at about 0.75-0.85 wt %, or at about 0.8 wt %.

50. The protein extrudate product of claim 33, wherein the protein extrudate product is in the form of a puff food product.

51. The protein extrudate product of claim 33, wherein the protein extrudate product is shaped in a geometric shape and size suitable for ease of consuming and/or handling.

52. The protein extrudate product of claim 33, wherein the protein extrudate product is fast orally dissolving.

53. The protein extrudate product of claim 33, wherein the protein extrudate product is protein-rich.

54. An edible food comprising the protein extrudate product of to any one of claims 33-53.

55. The edible food of claim 54, wherein the edible food comprises a puff food product, a puffed and coated food product, and/or a food product of different shapes and sizes suitable for oral consumption.
